(19) United States
(12) Patent Application Publication
(10) Pub. No.: US 2026/0247781 A1
(43) Pub. Date: Aug. 20, 2026

(54) HOLE-SELECTIVE CONTACT MATERIAL, PHOTOELECTRIC CONVERSION DEVICE AND POLYCYCLIC AROMATIC HYDROCARBON COMPOUND

(71) Applicant: OKINAWA INSTITUTE OF SCIENCE AND TECHNOLOGY SCHOOL CORPORATION, Kunigami-gun Okinawa (JP)

(72) Inventors: Yabing QI, Kunigami-gun, Okinawa (JP); Akimitsu NARITA, Kunigami-gun, Okinawa (JP); Tianhao WU, Kunigami-gun, Okinawa (JP); Xiushang XU, Kunigami-gun, Okinawa (JP); Ryota KABE, Kunigami-gun, Okinawa (JP); Luis Katsuya ONO, Kunigami-gun, Okinawa (JP); Silvia MARIOTTI, Kunigami-gun, Okinawa (JP); Saurav RAJ, Kunigami-gun, Okinawa (JP); Rengo YOSHIOKA, Kunigami-gun, Okinawa (JP)

(73) Assignee: OKINAWA INSTITUTE OF SCIENCE AND TECHNOLOGY SCHOOL CORPORATION, Kunigami-gun Okinawa (JP)

(21) Appl. No.: 19/159,804

(22) PCT Filed: Feb. 27, 2024
(86) PCT No.: PCT/JP2024/007179

§ 371 Date: Aug. 26, 2025

(30) Foreign Application Priority Data
Feb. 28, 2023 (JP) 2023-030557

(51) Int. Cl. H10K 30/40; H10K 30/86; H10K 85/60

(52) U.S. Cl. CPC H10K 30/40; H10K 30/86; H10K 85/624; H10K 85/633

(57) ABSTRACT

Provided is a hole-selective contact material for long-term stable PSCs. A hole-selective contact material comprises a polycyclic aromatic hydrocarbon compound, wherein the polycyclic aromatic hydrocarbon compound comprises a structure having a plurality of aromatic hydrocarbon rings, each of the aromatic hydrocarbon rings being fused with at least one other of the aromatic hydrocarbon rings, and at least one anchor group attached to the structure directly or via a linking group.

### TECHNICAL FIELD

The present disclosure relates to hole-selective contact material, photoelectric conversion device, and a polycyclic aromatic hydrocarbon compound.

### BACKGROUND

Perovskite solar cells (PSCs) have emerged as a promising photovoltaic owing to the unique optoelectronic properties of metal halide perovskite materials, including the strong light-absorption coefficient in the visible-light region, long diffusion length for charge carriers, and solution-based processibility suitable for the large-area solar modules. In recent years, inverted p-i-n structured PSCs with good compatibility for tandem solar cells and lower sensitivity to moisture and oxygen compared to that of the regular n-i-p devices, have made great improvements in terms of power conversion efficiency (PCE). This large efficiency progress could be attributed to the careful design of interface passivation layer, optimization of perovskite compositions, and development of novel hole-selective contacts. Among them, the design of hole-selective contacts at the device illumination side has a strong influence on not only the efficiency but also the long-term operational stability of inverted PSCs. Such design is currently under intensive investigation.

P-type organic semiconductors with low-temperature processibility and tunable energy-level for improved alignment with the perovskite layer have been frequently used as the hole-selective contacts for inverted PSCs. So far, many aromatic systems with the electron-donating ability, such as triarylamines, carbazoles, and polythiophene, have been employed as the π-conjugated building blocks of hole-selective molecules.

Poly(bis(4-phenyl)(2,4,6-trimethylphenyl)amine) (PTAA) is a commonly-used triarylamine-based hole-selective contact to produce high-performance inverted PSCs (NPLs 1 and 2).

PTAA derivative functionalized with pyridine anchoring groups (p-PY) as hole-selective contact has been investigated (NPL 3).

Additionally, the carbazole-based molecules such as 2PACz and MeO-2PACz with a phosphonic acid anchoring group have been investigated as a conformal hole-selective contact to produce the inverted PSCs with high efficiency (NPL 4).

A hole-selective molecule, EADR04, with a π-conjugated linker between the carbazole core and the anchoring group has been also researched (NPL 5).

Furthermore, a donor-acceptor (D-A) conjugated structure for hole-selective molecules has been investigated in order to improve their intrinsic photostability (NPL 6).

### CITATION LIST

### Non-Patent Literature

NPL 1: Y. Wang, L. Duan, M. Zhang, Z. Hameiri, X. Liu, Y. Bai, X. Hao, Sol. RRL 2022, 6, 2200234.
NPL 2: T. Wu, L. K. Ono, R. Yoshioka, C. Ding, C. Zhang, S. Mariotti, J. Zhang, K. Mitrofanov, X. Liu, H. Segawa, R. Kabe, L. Han, Y. B. Qi, Energy Environ. Sci. 2022, 15, 4612.
NPL 3: R. Chen, S. Liu, X. Xu, F. Ren, J. Zhou, X. Tian, Z. Yang, X. Guanz, Z. Liu, S. Zhang, Y. Zhang, Y. Wu, L. Han, Y. B. Qi, W. Chen, Energy Environ. Sci. 2022.
NPL 4: A. A1-Ashouri, A. Magomedov, M. Roβ, M. Jošt, M. Talaikis, G. Chistiakova, T. Bertram, J. A. Márquez, E. Köhnen, E. Kasparavičius, S. Levcenco, L. Gil-Escrig, C. J. Hages, R. Schlatmann, B. Rech, T. Malinauskas, T. Unold, C. A. Kaufmann, L. Korte, G. Niaura, V. Getautis, S. Albrecht, Energy Environ. Sci. 2019, 12, 3356.
NPL 5: E. Aktas, N. Phung, H. Köbler, D. A. González, M. Méndez, I. Kafedjiska, S.-H. Turren-Cruz, R. Wenisch, I. Lauermann, A. Abate, E. Palomares, Energy Environ. Sci. 2021, 14, 3976.
NPL 6: S. Zhang, R. Wu, C. Mu, Y. Wang, L. Han, Y. Wu, W.-H. Zhu, ACS Materials Letters 2022, 4, 1976.

### SUMMARY

### Technical Problem

Although great effort has been made, the operational lifetime of inverted PSCs is still lower than that of their regular counterparts at the current stage. Therefore, a new design concept for hole-selective contacts in terms of high photostability and good compatibility for solar modules is urgently needed.

### Solution to Problem

As a result of intensive study, the present inventors have found a novel strategy to design photostable and scalable hole-selective contact material based on a polycyclic aromatic hydrocarbon compound with regards to long-term stable PSCs and, in particular, long-term stable inverted PSCs.

The gist of the present disclosure is as follows:

[1] A hole-selective contact material comprising: a polycyclic aromatic hydrocarbon compound,
wherein the polycyclic aromatic hydrocarbon compound comprises a structure having a plurality of aromatic hydrocarbon rings, each of the aromatic hydrocarbon rings being fused with at least one other of the aromatic hydrocarbon rings, and at least one anchor group attached to the structure directly or via a linking group.

[2] The hole-selective contact material according to [1], wherein the anchor group is selected from the group consisting of a carboxylic acid group, a carboxylate group, a cyanoacrylic acid group, a cyanoacrylate group, a pyridine group, a phosphonic acid group, a tetracyanate group, a perylene dicarboxylic acid anhydride group, a 2-hydroxybenzonitrile group, an 8-hydroxyquinoline group, a pyridine-N-oxide group, a 3-hydroxy-N-methylpyridinium group, a catechol group, a hydroxamate group, a sulfonic acid group, an acetylacetonate group, a boronic acid group, a nitro group, a tetrazol group, a rhodamine group, a rhodamine-3-acetic acid group, a salicylic acid group, an aldehyde group, a carbamate group, an unsubstituted or substituted amino group, an unsubstituted or substituted ammonium group, a halogen atom, and an imine group.

[3] The hole-selective contact material according to [1] or [2], wherein the linking group is selected from the group consisting of an alkylene group, an alkenylene group, an alkinylene group, a cycloalkylene group, an arylene group, and a heteroarylene group.

[4] The hole-selective contact material according to any one of [1] to [3], wherein the structure having a plurality of aromatic hydrocarbon rings has an absorption edge from 350 to 550 nm.

[5] The hole-selective contact material according to [4], wherein the structure having a plurality of aromatic hydrocarbon rings has an absorption edge from 400 to 500 nm.

[6] The hole-selective contact material according to any one of [1] to [5], wherein the plurality of aromatic hydrocarbon rings is 3 to 16 aromatic hydrocarbon rings.

[7] The hole-selective contact material according to any one of [1] to [6], wherein the plurality of aromatic hydrocarbon rings is a plurality of benzene rings.

[8] The hole-selective contact material according to any one of [1] to [7], wherein the structure is benzo[rst]pentaphene.

[9] The hole-selective contact material according to any one of [1] to [8], wherein the polycyclic aromatic hydrocarbon compound comprises at least one solubility-enhancing group.

[10] The hole-selective contact material according to [9], wherein the solubility-enhancing group is selected from the group consisting of an alkyl group, an alkoxy group, an aryl group and an ester group.

[11] A photoelectric conversion device comprising the hole-selective contact material according to any one of claims [1] to [10].

[12] The photoelectric conversion device according to [11], wherein the photoelectric conversion device is a perovskite solar cell.

[3] A polycyclic aromatic hydrocarbon compound represented by formula (1):

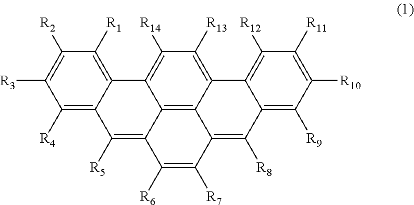

wherein
R₁, R₂, R₃, R₄, R₉, R₁₀, R₁₁, and R₁₂ are independently selected from hydrogen atoms, C1-C12 alkyl and C1-C12 alkoxy,
R₅ and R₈ are independently selected from Rₐ and -arylene-Rₐ, wherein Rₐ is carboxylic acid group, carboxylate group or carbamate group and
R₆, R₇, R₁₃, and R₁₄ are hydrogen atoms.

### Advantageous Effects

According to the present disclosure, a hole-selective contact material with regards to long-term stable PSCs and, in particular, long-term stable inverted PSCs, is provided. Certain hole-selective contact materials according to the present disclosure enable a fast and uniform hole extraction process and stabilize the organic components at the perovskite buried interface when used in PSCs. These advantages lead to a higher device efficiency than that of the frequently-used triphenylamine and carbazole-based hole-selective contacts.

### BRIEF DESCRIPTION OF THE DRAWINGS

In the accompanying drawings:

FIG. 1A: ¹H NMR spectrum (400 MHz, 298 K) of BPP in CD₂Cl₂.

FIG. 1B: ¹³C NMR spectrum (101 MHz, 298 K) of BPP in CD₂Cl₂.

FIG. 2A: ¹H NMR spectrum (400 MHz, 298 K) of SA-BPP in CD₂Cl₂.

FIG. 2B: ¹³C NMR spectrum (101 MHz, 298 K) of SA-BPP in CD₂Cl₂.

FIG. 3A: High-resolution mass spectra of BPP.

FIG. 3B: High-resolution mass spectra of SA-BPP (Top: calculated isotopic distribution pattern; down: experimental isotopic distribution pattern).

FIG. 4: ¹H NMR spectrum (400 MHz, 298 K) of BPP-Boc in CD₂Cl₂.

FIG. 5: HOMO and LUMO orbital distributions using the DFT method in Gaussian 09 program.

FIG. 6A: Properties of the BPP and SA-BPP molecules:

Chemical structures of the BPP and SA-BPP molecules, and the schematic illumination of the SA-BPP hole-selective contact that could form the bonding with both ITO substrate and perovskite layer in the inverted PSCs.

FIG. 6B: FTIR spectra of the pure SA-BPP, SA-BPP-ITO mixture, and SA-BPP-PbI₂ mixture.

FIG. 6C: Band structures of the BPP and SA-BPP films coated on ITO substrate determined by the UPS and LEIPS measurements.

FIG. 6D: Energy offset between the HOMO of BPP and SA-BPP, and the VBM of perovskite absorber, the red dash line indicates the EF position.

FIG. 7A: The Hole-Selective Contacts and Subsequently-Deposited Perovskite Films: Surface potential mapping profile on a 50 μm×50 μm region for the ITO/MeO-2PACz.

FIG. 7B: Surface potential mapping profile on a 50 μm×50 μm region for ITO/BPP.

FIG. 7C: Surface potential mapping profile on a 50 μm×50 μm region for ITO/SA-BPP samples. The potential statistical distributions are also shown to indicate the homogeneity of these thin films.

FIG. 7D: Top-view SEM image of the perovskite film deposited on MeO-2PACz.

FIG. 7E: Top-view SEM image of the perovskite film deposited on BPP.

FIG. 7F: Top-view SEM image of the perovskite film deposited on SA-BPP hole-selective contacts.

FIG. 7G: Cross-section SEM image of the perovskite film deposited on MeO-2PACz.

FIG. 7H: Cross-section SEM image of the perovskite film deposited on BPP.

FIG. 7I: Cross-section SEM image of the perovskite film deposited on SA-BPP hole-selective contacts.

FIG. 8A: Surface potential mapping profile on a 50 μm×50 μm region for the perovskite film deposited on the MeO-2PACz substrate.

FIG. 8B: Surface potential mapping profile on a 50 μm×50 μm region for the perovskite film deposited on the BPP substrate.

FIG. 8C: Surface potential mapping profile on a 50 μm×50 μm region for the perovskite films deposited on the SA-BPP substrate.

FIG. 8D: Grain size distribution plots of the corresponding perovskite film.

FIG. 8E: Grain size distribution plots of the corresponding perovskite film.

FIG. 8F: Grain size distribution plots of the corresponding perovskite film.

FIG. 9: XRD patterns of the perovskite films grown on different hole-selective contacts. Diffraction peaks at 14.0°, 20.1°, 23.8°, 28.1°, and 32.0° are associated with the (100), (110), (111), (200), and (210) planes of the perovskite phase, respectively.

FIG. 10A: Carrier Dynamics at the Hole-Selective Contact/Perovskite Interface: Delay time-dependent transient absorption spectra at the pump-probe delay time of 30-500 ps for the MeO-2PACz/perovskite film deposited on the glass substrate.

FIG. 10B: Delay time-dependent transient absorption spectra at the pump-probe delay time of 30-500 ps for the BPP/perovskite film deposited on the glass substrate.

FIG. 10C: Delay time-dependent transient absorption spectra at the pump-probe delay time of 30-500 ps for the SA-BPP/perovskite film deposited on the glass substrate.

FIG. 10D: PL mapping profile on a 100 μm×100 μm region of the perovskite film deposited on the MeO-2PACz substrate.

FIG. 10E: PL mapping profile on a 100 μm×100 μm region of the perovskite film deposited on the BPP substrate.

FIG. 10F: PL mapping profile on a 100 μm×100 μm region of the perovskite film deposited on the SA-BPP substrate.

FIG. 11A: Transient absorption dynamics of the ground-state bleaching peaks of the perovskite film with the contact of MeO-2PACz on glass substrate.

FIG. 11B: Transient absorption dynamics of the ground-state bleaching peaks of the perovskite film with the contact of BPP on glass substrate.

FIG. 11C: Transient absorption dynamics of the ground-state bleaching peaks of the perovskite film with the contact of SA-BPP on glass substrate.

FIG. 12A: Total DOS plots of the defect-containing FAPbI₃ (100) slabs without the passivation of BPP and SA-BPP, trap states at the band edge were also indicated.

FIG. 12B: Total DOS plots of the defect-containing FAPbI₃ (100) slabs with the passivation of BPP, trap states at the band edge were also indicated.

FIG. 12C: Total DOS plots of the defect-containing FAPbI₃ (100) slabs with the passivation of SA-BPP, trap states at the band edge were also indicated. The bottom panels show the HOMO charge distribution plots of the systems.

FIG. 13A: Photostability of the Hole-selective Contacts and Their Relative Interfaces with the Perovskite Layer: High-resolution XPS spectra of the C 1s region for the MeO-2PACz solid-state film coated on ITO substrate before and after being exposed to simulated 1-sun light for 100 hours in dry N₂.

FIG. 13B: High-resolution XPS spectra of the C Is region for the SA-BPP solid-state film coated on ITO substrate before and after being exposed to simulated 1-sun light for 100 hours in dry N₂.

FIG. 13C: High-resolution XPS spectra of the C 1s region for the PTAA solid-state film coated on ITO substrate before and after being exposed to simulated 1-sun light for 100 hours in dry N₂.

FIG. 13D: In situ MS contour profile (m/z=1-200 amu) of the MeO-2PACz/perovskite film coated on the ITO glass.

FIG. 13E: In situ MS contour profile (m/z=1-200 amu) of SA-BPP/perovskite film coated on the ITO glass.

FIG. 13F: In situ MS contour profile (m/z=1-200 amu) of PTAA/perovskite film coated on the ITO glass. The simulated 1-sun light was applied after 15 min of MS measurement and the total illumination time was 120 min.

FIG. 13G: Possible degradation mechanisms at the perovskite/hole-selective contact interface under 1-sun illumination condition.

FIG. 14A: Current mapping profile of the fresh ITO/MeO-2PACz film.

FIG. 14B: Current mapping profile of ITO/SA-BPP film.

FIG. 14C: Current mapping profile of the fresh ITO/PTAA film.

FIG. 14D: the ITO/MeO-2PACz film after being aged under continuous sun illumination for 100 hours in a N₂ atmosphere, the applied bias is fixed at 1.0 V.

FIG. 14E: the ITO/SA-BPP film after being aged under continuous sun illumination for 100 hours in a N₂ atmosphere, the applied bias is fixed at 1.0 V.

FIG. 14F: the ITO/PTAA film after being aged under continuous sun illumination for 100 hours in a N₂ atmosphere, the applied bias is fixed at 1.0 V. It is worth noting that the hole-selective contacts used for current mapping measurement are thicker than those used for fabricating the inverted PSCs to better understand the conductivity change under operational condition.

FIG. 15A: Schematic diagram of our home-designed in situ mass spectrometry (MS) system for investigating the light-induced degradation at the hole-selective contact/perovskite interface.

FIG. 15B: Partial pressure of the released gas products versus illumination time from the perovskite film deposited on MeO-2PACz substrate.

FIG. 15C: Partial pressure of the released gas products versus illumination time from the perovskite film deposited on SA-BPP substrate.

FIG. 15D: Partial pressure of the released gas products versus illumination time from the perovskite film deposited on PTAA substrate.

FIG. 16A: Electrostatic potential (ESP) mapping profiles of the MeO-2PACz, SA-BPP, and the PTAA molecules containing three triarylamine units calculated by the DFT method.

FIG. 16B: Electron deformation density plots of the MeO-2PACz, SA-BPP, and the PTAA monomer interacted with a FA+ cation, the cation-π interaction energies were calculated to be −0.47 eV, −1.08 eV, and −0.28 eV for the MeO-2PACz, SA-BPP, and PTAA, respectively.

FIG. 17A: Photovoltaic Performance of Inverted Perovskite Solar Cells and Modules: Cross-section SEM image of the SA-BPP-based inverted PSCs.

FIG. 17B: J-V curves of the small-sized inverted PSCs (champion device) based on the MeO-2PACz, BPP, and SA-BPP hole-selective contacts.

FIG. 17C: EQE spectra of the MeO-2PACz and SA-BPP-based champion devices.

FIG. 17D: Photographs of the 5 cm×5 cm solar module based on SA-BPP hole-selective contact.

FIG. 17E: J-V curves of the SA-BPP-based solar module on an aperture area of 22.4 cm², the inset shows the module architecture with P1, P2, and P3 patterns.

FIG. 17F: PL intensity mapping profile of the perovskite film coated on the 5 cm×5 cm ITO (with P1 pattern)/SA-BPP substrate.

FIG. 18A: Designed patterns of the solar module on a 5 cm×5 cm ITO substrate.

FIG. 18B: Optical microscope image of the P1, P2, and P3 patterns, indicating a geometrical fill factor (GFF) of 95.8%.

FIG. 19A: Operational stability test system for the PSCs. The temperature of testing device is monitored by a thermocouple (blue line), and a continuous N₂ flow was put into the sample chamber during entire operational process.

FIG. 19B: The solar spectrum generated by xenon white light source used for the stability measurement.

FIG. 20A: Operational Stability of Inverted Perovskite Solar Cells and Modules: Operational stability tests of the champion devices in a N₂-filled chamber at 45° C. for the small-sized solar cells measured under a constant bias close to the initial maximum power point.

FIG. 20B: Operational stability tests of the champion devices in a N₂-filled chamber at 45° C. for the 5 cm×5 cm solar modules measured under a constant bias close to the initial maximum power point.

FIG. 21A: Cross-section SEM image of the PTAA-based inverted PSCs.

FIG. 21B: J-V curves of the small-sized inverted PSCs based on a PTAA hole-selective contact for the operational stability measurement.

FIG. 22A: XPS high-resolution O 1s spectra of the PCBM/C₆₀ films without and with the ALD-SnO₂ protection layer.

FIG. 22B: XPS high-resolution Sn 3d spectra of the PCBM/C60 films without and with the ALD-SnO₂ protection layer.

FIG. 22C: Optical microscopy images of the solar modules (P1-P3 patterns) without/with ALD-SnO₂ protection layer before and after exposed to simulated 1-sun light for 100 hours, the length of scale bar is 50 μm.

FIG. 23A: Forward and reverse J-V curves of the 0.1 cm² inverted PSCs based on the BPP—Br hole-selective contact.

FIG. 23B: Operational stability tests of the 0.1 cm² inverted PSCs in a N₂-filled chamber at 45° C. based on the BPP—Br hole-selective contact.

FIG. 24A: Forward and reverse J-V curves of the 0.1 cm² inverted PSCs based on the BPP-MeOPA hole-selective contact.

FIG. 24B: Operational stability tests of the 0.1 cm² inverted PSCs in a N₂-filled chamber at 45° C. based on the BPP-MeOPA hole-selective contact.

FIG. 25A: Forward and reverse J-V curves of the 0.1 cm² inverted PSCs based on the BPP-Boc hole-selective contact.

FIG. 25B: Operational stability tests of the 0.1 cm² inverted PSCs in a N₂-filled chamber at 45° C. based on the BPP-Boc hole-selective contact.

### DETAILED DESCRIPTION

The presently disclosed techniques will be described in detail below.

### (Terms)

“Alkyl group” may be linear or branched, and the number of carbon atoms may generally be 1 to 20. “Alkyl group” includes methyl, ethyl, n-propyl, isopropyl, n-butyl, sec-butyl, tert-butyl, n-pentyl, sec-pentyl, tert-pentyl, neopentyl, amyl, hexyl, heptyl, octyl, nonyl, decyl, undecyl, dodecyl, etc.

“Alkenyl group” may be linear or branched, and the number of carbon atoms may generally be 2 to 20. “Alkenyl group” includes vinyl, allyl, butenyl, butadienyl, pentenyl, hexenyl, heptenyl, octenyl, nonenyl, decenyl, undecenyl, dodecenyl, etc.

“Alkynyl group” may be linear or branched, and the number of carbon atoms may generally be 2 to 20. “Alkynyl group” includes ethynyl, propargyl, butynyl, pentynyl, hexynyl, heptynyl, octynyl, nonynyl, decynyl, undecynyl, dodecynyl, etc.

“Cycloalkyl group” may be a monocyclic group or a polycyclic group, and the number of carbon atoms may generally be 3 to 30. “Cycloalkyl group” includes cyclopentyl, cyclohexyl, cycloheptyl, adamantyl, methylcyclohexyl, ethylcyclohexyl, etc.

“Alkylene group” may be linear or branched, and the number of carbon atoms may generally be 1 to 20. “Alkylene group” includes methylene, ethylene, propylene, butylene, pentylene, hexylene, heptylene, octylene, nonylene, decylene, undecylene, dodecylene, etc.

“Alkenylene group” may be linear or branched, and the number of carbon atoms may generally be 2 to 20 carbon atoms. “Alkenylene group” includes vinylene, propenylene, butenylene, butadienylene, octenylene, decenylene, dodecenylene, etc.

“Alkynylene group” may linear or branched, and the number of carbon atoms may generally be 2 to 20 carbon atoms. “Alkynylene group” includes ethynylene, propynylene, butynylene, pentynylene, hexynylene, heptynylene, octynylene, noninylene, decynylene, undecinylene, dodecinylene group, etc.

“Alkoxy group” is an alkyl-O—, and the description of the alkyl group above applies to the alkyl moiety. “Alkoxy group” includes methoxy, ethoxy, propoxy, isopropoxy, n-butoxy, sec-butoxy, tert-butoxy, allyloxy, etc.

“Cycloalkylene group” is a divalent group corresponding to a cycloalkyl group.

“Aryl group” is a monovalent aromatic hydrocarbon group, and the number of carbon atoms may generally be 6 to 20 carbon atoms. “Aryl group” includes phenyl, naphthyl, biphenyl, phenanthryl, etc.

“Heteroaryl group” is a monovalent aromatic heterocyclic group, and the number of ring atoms may generally be 4 to 10. Hetero atoms may be nitrogen, oxygen and sulfur. “Heteroaryl group” includes pyridinyl, furanyl, thiophenyl, etc.

“Arylene group” is a divalent group corresponding to an aryl group.

“Heteroarylene group” is a divalent group corresponding to a heteroaryl group.

“Halogen atom” involves fluorine, chlorine, bromine and iodine atom.

### (Structure Having a Plurality of Aromatic Hydrocarbon Rings)

Certain hole-selective contact materials according to the present disclosure comprise a polycyclic aromatic hydrocarbon compound. The polycyclic aromatic hydrocarbon compound comprises a structure having a plurality of aromatic hydrocarbon rings. Each of the aromatic hydrocarbon rings is fused with at least one other of the aromatic hydrocarbon rings. That is, each of the aromatic hydrocarbon rings shares two neighboring carbon atoms with at least one other of the aromatic hydrocarbon rings. Certain structures are advantageous in obtaining tunable optoelectronic properties and stability against light and heat.

A structure in which all of the aromatic hydrocarbon rings are benzene rings may correspond to a partial structure which is included in a single-layer graphene. Such a structure may also be referred to as a graphene-like conjugated molecule or graphenoid. The graphene-like conjugated structure is advantageous in exhibiting charge carrier mobility due to its electron delocalization character that results from the large I-extended conjugation structure.

The number of aromatic hydrocarbon rings is one or more but preferably two or more. The number of aromatic hydrocarbon rings may be 16 or less. An optical absorption edge of the structure having a plurality of aromatic hydrocarbon rings may be 700 nm or less. From the viewpoint of ensuring a suitable optical absorption edge, preferably in the range from 350 to 550 nm, the number of aromatic hydrocarbon rings is preferably, 3 to 16, and more preferably, 4 to 12, and even more preferably, 4 to 8. The absorption edge from 350 to 550 nm, corresponding to the optical energy gap of 2.25-3.54 eV, is preferred with respect to the structure having a plurality of aromatic hydrocarbon rings for the following reason. The electronic energy gap, which can be proportional to optical energy gap for the structure, should be small enough to secure the efficient charge transport through the hole-selective contact materials according to the present disclosure, while it should not be too small to avoid significant absorption of the solar light. The absorption edge from 400 to 500 nm is more preferred. For determination of the absorption edge, the method described in the following literature may be used.
J. C. S. Costa et al., “Optical band gaps of organic semiconductor materials”, Optical Materials, Volume 58, August 2016, Pages 51-60

Examples of structures where all aromatic hydrocarbon rings are benzene rings include anthracene, phenanthrene, pyrene, tetracene, chrysene, perylene, benzo[a]anthracene, benzo[a]pyrene, dibenzo[a,h]anthracene, benzo[ghi]perylene, pentaphene, benzo[rst]pentaphene, coronene, anthanthrene, triphenylene, trinaphthylene, hexa-peri-hexabenzocoronene, naphtho[8,1,2-bcd]perylene, naphtho[1,2,3,4-ghi]perylene, dibenzo[b,k]chrysene, benzo[c]picene, benzo[a]pentacene, benzo[a]coronene, phenanthrol [5,4,3,2-efghi]perylene, benzo[pqr]naphtho[8,1,2-bcd]perylene, benzo[vwx]hexaphene, benzo[c]naphtho[2,1,8-qra]tetracene, dibenzo[h,rst]pentaphene, naphtho[8, 1,2-abc]coronene, pyranthrene, tribenzo[de,kl,rst]pentaphene, tetrabenzo[de,hi,mn,qr]tetracene, tribenzo[f,k,m]tetraphene, dibenzo[a,c]pentacene, dibenzo[a,1]pentacene, heptaphene, ovalene, dinaphtho[2,1,8,7-defg: 2′, 1′,8′,7′-opqr]pentacene, dinaphtho[2,1,8,7-defg: 2′,1′,8′,7′-ijkl]pentaphene, dibenzo[a,j]coronene, naphtho[1,2-a]coronene, naphtho[8,1,2-hij]hexaphene, dibenzo[fg,ij]phenanthrol [2,1,10,9,8,7-pqrstuv]pentaphene, peryleno [3,2,1,12-pqrab]perylene, benzo[rst]dinaphtho[2,1,8,7-defg: 2′,1′,8′,7′-ijkl]pentaphene, dinaphtho[8,1,2-abc: 8′, 1′,2′-jkl]coronene, dinaphtho[8, 1,2-abc: 2′, 1′,8′-klm]coronene, naphth [2′, 1′,8′,7′: 4, 10]anthra [1,9,8-abcd]coronene, tetrabenzo[def,lm,qrs,yz]pyranthrene, hexabenzo[bc,ef,hi,kl,no,qr]coronene, tetrabenzo[bc, ef, o,uv]phenanthro [2,1,10,9-hijk]ovalene.

Certain structures may include a ring other than benzene ring. Examples of such structures include acenaphthylene, acenaphthene, fluorene, fluoranthene, benzo[b]fluoranthene, benzo[j]fluoranthene, benzo[k]fluoranthene, benzo[c]fluorene, indeno[1,2,3-cd]pyrene, corannulene.

The polycyclic aromatic hydrocarbons recited in the following literatures are also available for the structure of the present disclosure:
R. Rieger et al., “polycyclic aromatic hydrocarbons as model cases for structural and optical studies”, Journal of Physical Organic Chemistry, Volume 23, Issue 4 p. 315-325 (2010)
M. Kastler et al., “From Armchair to Zigzag Peripheries in Nanographenes”, J. Am. Chem. Soc. 2006, 128, 29, 9526-9534
J. Prakash et al., “Simultaneous Quantification of Multiple Polycyclic Aromatic Hydrocarbons in Aqueous Media using Micelle Assisted White Light Excitation Fluorescence”, Scientific Reports volume 10, Article number: 8921 (2020).

The substituted or unsubstituted polycyclic aromatic hydrocarbon compounds disclosed in WO2020/070085 are also available for the structures of the present disclosure.

The structures can be produced by the “DPEX” reaction described in the literatures below. The polycyclic aromatic hydrocarbon compounds disclosed in these references are also available for the structures of the present disclosure.
D. Lungerich, et al., “Dehydrative π-extension to nanographenes with zig-zag edges”, Nature Communications volume 9, Article number: 4756 (2018)
M. Feofanov, et al., “Modular Approach to the Synthesis of Two-Dimensional Angular Fused Acenes”, Org. Lett. 2020, 22, 5, 1698-1702.

The structures can be produced by the “APEX” reaction and cyclization of acetylenes described in the literatures below. The polycyclic aromatic hydrocarbon compounds disclosed in these references are also available for the structures of the present disclosure.
K. Ozaki et al., “One-shot K-region-selective annulative π-extension for nanographene synthesis and functionalization”, Nature Communications, volume 6, Article number: 6251 (2015).
Tse-An Chen, et al., “Synthesis of Polyaromatic Hydrocarbons from Bis(biaryl)diynes: Large PAHs with Low Clar Sextets”, Chemistry-A European Journal, Volume17, Issue29, 2011, Pages 8023-8027.

A structure with a combination of zigzag and armchair edges may be preferred in that it can provide an excellent platform to tune the energy levels. An example of such a structure with a combination of zigzag and armchair edges includes benzo[rst]pentaphene, but other structures may be used.

### (Anchor Groups)

The polycyclic aromatic hydrocarbon compound according to the present disclosure preferably comprises at least one anchor group attached to the structure directly or via a linking group. An anchor group is a group that acts as an anchor to the surroundings when the hole-selective contact material according to the present disclosure is used.

Examples of anchor groups include carboxylic acid group, carboxylate group, cyanoacrylic acid group, cyanoacrylate group, pyridine group, phosphonic acid group, tetracyanate group, perylene dicarboxylic acid anhydride group, 2-hydroxybenzonitrile group, 8-hydroxyquinoline group, pyridine-N-oxide group, 3-hydroxy-N-methylpyridinium group, catechol group, hydroxamate group, sulfonic acid group, acetylacetonate group, boronic acid group, nitro group, tetrazol group, rhodamine group, rhodamine-3-acetic acid group, salicylic acid group, aldehyde group, carbamate group, unsubstituted or substituted amino group, unsubstituted or substituted ammonium group, halogen atom, and imine group. These may be used alone or in combination of two or more.

The substituted amino group and substituted ammonium group include amino group and ammonium group having one or more substituent(s) such as an alkyl group and aryl that may be further substituted by for example alkoxy group. The substituted amino group includes dialkylamino, bis(methoxyphenyl)amino.

The linking group may be an alkylene group, an alkinylene group, a cycloalkylene group, an arylene group, and a heteroarylene group, and the combination of two or more of these.

The anchor group is preferably a carboxylic acid group, and a carboxylate group. The carboxylic acid group includes the group represented by —COOH, and may be in a form of a salt. The salt may be an inorganic salt or an organic salt. Examples of salts include an alkaline metal salt (for example, —COONa, —COOK, —COOCs), and an ammonium salt. The cation of the salt may be a quaternary ammonium, such as methylammonium, imidazolium, dimethylammonium, formamidinium, guanidinium, and aziridinium. The carboxylate group includes the group represented by —COOR, where R is an alkyl group or an aryl group, preferably methyl, ethyl, phenyl. It is preferred that these groups are attached to the structure directly or via an arylene group (preferably, a phenylene group).

It is preferred that the compound have one to eight anchor group(s). More preferably, the number of anchor group is two to six. In case two or more anchor groups are present, they may be the same or different. The anchor groups are preferably the same. The anchor groups may be attached to the same aromatic hydrocarbon ring or to different aromatic hydrocarbon rings. They are preferably attached to different aromatic hydrocarbon rings. In certain embodiments, where stronger anchoring is desirable, the polycyclic aromatic hydrocarbon compounds comprise two to four anchor groups. In certain embodiments, the polycyclic aromatic hydrocarbon compounds comprise one or two bidentate or polydentate anchor groups.

The anchor group can be introduced into the compound by known methods.

### (Solubility-Enhancing Groups)

Certain polycyclic aromatic hydrocarbon compounds according to the present disclosure may comprise solubility-enhancing group(s) attached to the structure in order to improve solubility of the compound in a solvent. Examples of solubility-enhancing groups include an alkyl group (preferably, C1-C₁₂ alkyl), an alkoxy group (preferably, C1-C₁₂ alkoxy), an ester group. These may be used alone or in combination of two or more. The ester group includes a group having —C(═O)O—, such as a carboxylate group.

The polycyclic aromatic hydrocarbon compounds may comprise one to four solubility enhancing group(s). In certain embodiments, the polycyclic aromatic hydrocarbon compounds comprise two solubility-enhancing groups. In certain embodiments, where greater solubility is desirable, the polycyclic aromatic hydrocarbon compounds comprise three or four solubility enhancing groups.

On the other hand, in cases where the anchor group also promotes solubility, sufficient solubility may be obtained without the additional solubility-enhancing group. For example, an anchor group having an alkyl group, an alkoxy group or an ester group may be mentioned.

Further, if the anchor group does not comprise an ester group or is a methyl carboxylate ester group or an ethyl carboxylate ester group, the solubility-enhancing group may be a C3-C10 alkyl carboxylate group. Examples of C3-C10 alkyl carboxylate groups include a t-Bu carboxylate group, a pentyl carboxylate group, a hexyl carboxylate group, a heptyl carboxylate group, and an octyl carboxylate group.

In case two or more anchor groups are present, the solubility-enhancing groups may be the same or different, but preferably they are the same.

The solubility-enhancing substituents can be introduced into the compound by known methods.

### (Other Substituents)

The polycyclic aromatic hydrocarbon compound according to the present disclosure may have substituents other than anchor group(s), linking group(s) with anchor group(s), and solubility-enhancing group(s).

### (Polycyclic Aromatic Hydrocarbon Compound)

Preferred compounds of the polycyclic aromatic hydrocarbon compound used in the hole-selective contact material according to the present disclosure include the compound represented by formula (1):

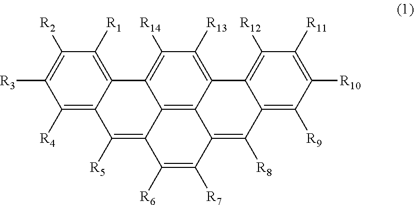

wherein
R₁ to R₁₄ are independently selected from hydrogen atom, an anchor group, a linking group with an anchor group, or a solubility enhancing group,
provided that at least one of which is an anchor group or a linking group substituted by an anchor group.

Examples of an anchor group, a linking group and a solubility enhancing group are mentioned above.

It is preferred that the anchor group and the soluble group are not too close from each other, for example, they are bonded to non-adjacent carbon atoms. The anchor group and the soluble group may be bonded to different hydrocarbon aromatic rings.

The compound represented by formula (1), wherein R₁, R₂, R₃, R₄, R₉, R₁₀, R₁₁ and R₁₂ are independently selected from hydrogen atoms and a solubility enhancing group, R₅ and R₅ are independently selected from an anchor group and a linking group substituted by an anchor group, and R₆, R₇, R₁₃, and R₁₄ are hydrogen atoms, is preferred.

For example, the compound represented by formula (1), wherein R₁, R₂, R₃, R₄, R₉, R₁₀, R₁₁ and R₁₂ are independently selected from hydrogen atoms, C1-C12 alkyl and C1-C12 alkoxy, R₅ and R₈ are independently selected from Rₐ and -arylene-Rₐ, wherein Rₐ is carboxylic acid group, carboxylate group, or carbamate group, unsubstituted or substituted amino group or halogen atom, and R₆, R₇, R₁₃, and R₁₄ are hydrogen atoms, may be mentioned.

The compound represented by formula (1), wherein R₁, R₂, R₃, R₄, R₉, R₁₀, R₁₁ and R₁₂ are independently selected from hydrogen atoms, C1-C₁₂ alkyl and C1-C12 alkoxy, R₅ and R₈ are independently selected from Rₐ and -arylene-Rₐ, wherein Rₐ is carboxylic acid group, carboxylate group, or carbamate group, and R₆, R₇, R₁₃, and R₁₄ are hydrogen atoms, is more preferred.

The compound represented by formula (1), wherein each of R₂ and R₁₁ is a solubility enhancing substituent, and each of R₅ and R₈ is an anchor group or a linking group substituted by an anchor group, and each of R₁, R₃, R₄, R₆, R₇, R₉, R₁₀, R₁₂, R₁₃, and R₁₄ is a hydrogen atom, is also preferred.

For example, the compound represented by formula (1), wherein R₂ and R₁₁ are independently selected from C1-C₁₂ alkyl and C1-C₁₂ alkoxy, R₅ and R₈ are independently selected from Rₐ and -arylene-Rₐ, wherein Rₐ is carboxylic acid group, carboxylate group, carbamate group, unsubstituted or substituted amino group or halogen atom, and R₁, R₃, R₄, R₆, R₇, R₉, R₁₀, R₁₂, R₁₃, and R₁₄ are hydrogen atoms, may be mentioned.

The compound represented by formula (1), wherein R₂ and R₁₁ are independently selected from C1-C12 alkyl and C1-C12 alkoxy, R₅ and R₈ are independently selected from Rₐ and -arylene-Rₐ, wherein Rₐ is carboxylic acid group, carboxylate group, or carbamate group, and R₁, R₃, R₄, R₆, R₇, R₉, R₁₀, R₁₂, R₁₃, and R₁₄ are hydrogen atoms, is more preferred.

### (Absorption Edge)

An optical absorption edge of the polycyclic aromatic hydrocarbon compound in the present disclosure may be 700 nm or less. It is preferred that the polycyclic aromatic hydrocarbon compound in the present disclosure has an absorption edge of from 350 to 550 nm, more preferably, from 400 to 500. By controlling the absorption edge within this range, the loss of light absorption caused by the compound can be suppressed, and high photoelectric conversion efficiency of the hole-selective contact material according to the present disclosure can be expected. The absorption edge can be controlled by, for example, changing the number of aromatic hydrocarbon rings and substituents included in the compound. Since a layer of the hole-selective contact in PSC is usually very thin, the polycyclic aromatic hydrocarbon compound is allowed to have an upper absorption edge of 550 nm.

### (Solubility)

Certain polycyclic aromatic hydrocarbon compounds according to the present disclosure tend to have excellent solubility in a solvent. Thus, certain hole-selective contact materials according to the present disclosure can be used in wet processes such as spin coating, screen printing, dipping, and can be easily used in the manufacture of photoelectronic conversion devices. Solubility can be controlled by, for example, changing the number of aromatic hydrocarbon rings and substituents included in the compound.

### (Hole-Selective Contact Material)

The hole-selective contact material according to the present disclosure preferably exhibits fast and uniform hole extraction when used in photoelectric conversion devices including PSCs. In particular, the hole-selective contact material according to the present disclosure preferably stabilizes the organic components at the perovskite buried interface when used in PSCs.

### (Photoelectronic Conversion Device)

The hole-selective contact material according to the present disclosure can be used in various photoelectronic conversion devices. Examples of photoelectronic conversion devices include optical sensors, optical diodes, and solar cells such as PSCs.

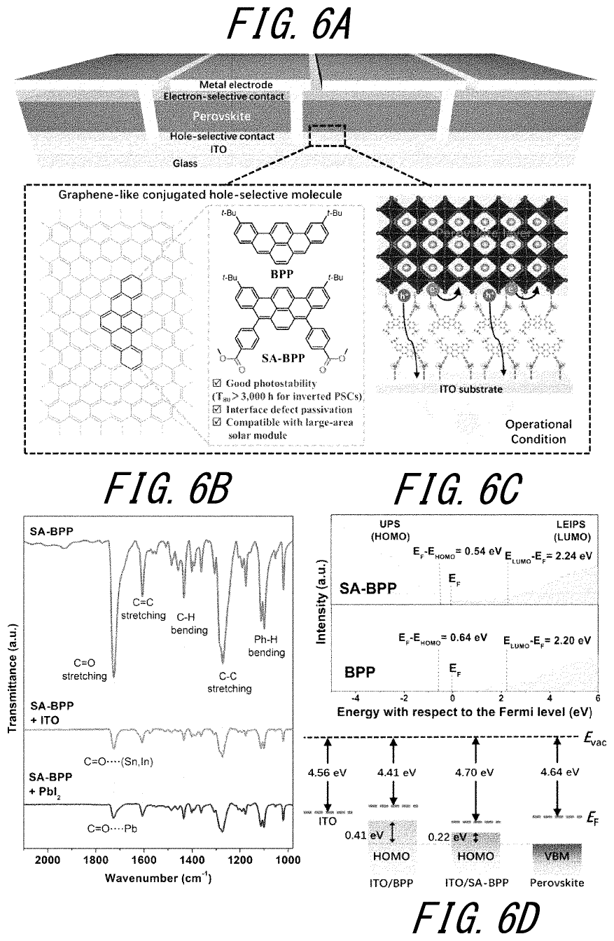

APSC includes an inverted PSC as shown in FIG. 6A. Typically, an inverted PSC has a substrate (glass in FIG. 6A), a transparent electrode (ITO in FIG. 6A), a hole-selective contact layer, a perovskite layer (light absorption layer), an electron-selective contact layer, and a back electrode (metal electrode in FIG. 6A) in this order. In addition to the above, an inverted PSC may include any known functional layer. The hole-selective contact material according to the present disclosure is suitable for forming the hole-selective contact layer.

The method of manufacturing PSCs using the hole-selective contact material according to the present disclosure is not particularly limited. For example, the following methods can be used.

The hole-selective contact material according to the present disclosure can be provided in the form of a solution in which a specific polycyclic aromatic hydrocarbon compound is dispersed or dissolved in a solvent. The solution can be applied to the surface of the transparent electrode and dried to form a hole-selective contact layer. The method of application is not particularly limited and includes spin coating, screen printing, dipping.

A perovskite layer can be formed on top of the hole-selective contact layer by using any known method. Material used to form the perovskite layer is not particularly limited and preferably contains at least one perovskite compound indicated by formula: ABX₃.

The electron-selective contact layer can be formed on top of the perovskite layer by using any known method. Material used to form the electron-selective contact layer is not particularly limited, and semiconductor materials such as silicon, germanium, metal chalcogenides can be used.

The back electrode can be formed on top of the electron-selective contact layer by using any known method. Material used to form the back electrode is not particularly limited, and conductive materials such as metals, carbon compounds, conductive metal oxides, and conductive polymers can be used.

Optional functional layers include a compact layer to improve photoelectric conversion efficiency and can be provided between the transparent electrode and the hole-selective contact layer or between the back electrode and the electron-selective contact layer. Material used to form the compact layer is not particularly limited, and metal oxides such as TiO₂, TiOₓ, SnO₂, ZnO₂ may be used. The compact layer can be formed by using any known method such as sputtering or ALD (Atomic Layer Deposition), etc.

The hole-selective contact material according to the present disclosure can be used for forming a hole-selective contact layer in planar PSCs or mesoporous PSCs.

### Examples

The following provides a more specific description of the present disclosure based on examples.

The materials and analytical methods used in the experiments are as follows.

### Example 1 (Synthesis and Characterization)

### Materials

All chemicals were used without further purification, including PbI2 (99.99%, TCI), MAI (GreatCell Solar), MACI (GreatCell Solar), FAI (GreatCell Solar), [6,6]-phenyl-C61-butyric acid methyl ester (PCBM, TCI), fullerene C₆₀ (TCI), bathocuproine (BCP, TCI), MeO-2PACz (TCI), poly(bis(4-phenyl) (2,4,6-trimethylphenyl)amine) (PTAA, TCI), (4-(methoxycarbonyl)phenyl) boronic acid (TCI), tris(dibenzylideneacetone)dipalladium(0)(Pd2(dba)3) (Sigma-Aldrich), 2-dicyclohexylphosphino-2′,6′-dimethoxybiphenyl(SPhos) (Sigma-Aldrich), K₃PO₄ (Sigma-Aldrich), MgSO4 (Wako), toluene (Wako), ethyl acetate (99.9%, Sigma-Aldrich, EtOAc, Wako), chlorobenzene (99.8%, Wako), ethanol (Wako), isopropanol (Wako), N,N-dimethylformamide (DMF, Wako), and dimethyl sulfoxide (DMSO, Wako), hexane (Wako), CH₂Cl₂ (Wako).

### (Sample Characterization)

Liquid-state Nuclear Magnetic Resonance (NMR) spectra were recorded in CD₂Cl₂, and CDCl₃ using Bruker DPX 400 NMR spectrometers. Chemical shifts (8) were expressed in ppm relative to the residual of solvents (CD₂Cl₂, ¹H: 5.32 ppm, ¹³C: 54.00 ppm). Coupling constants (J) were recorded in Hertz. Abbreviations: s=singlet, d=doublet, t=triplet.

High-resolution mass spectra (HRMS) were recorded on a Thermo Scientific LTQ-Orbitrap Electrospray Ionization (ESI) Mass Spectrometer.

The XPS and UPS spectra were recorded by Kratos X-ray photoelectron spectrometer (AXIS Ultra HAS) measured with the monochromatic A1 Kα (1486.6 eV) source and non-monochromatic He 1α (21.22 eV) sources, respectively. The XPS curves were fitted by Gaussian-Lorentzian functions after removing the inelastic scattering processes.

Scanning Kelvin probe microscopy (SKPM) measurements were performed by an atomic force microscope (AFM, Asylum Research MFP-3D, Oxford Instruments) using Ti/Ir coated Si cantilevers with a nominal spring constant of 2.8 Nm⁻¹ (Model: ASYELEC-01-R2), a Nap mode was applied to determine the potential difference between the AFM tip with a DC bias voltage and the sample surface.

The conductive AFM measurements were performed by the same AFM equipment (Contact mode) using the Cr/Pt coated Si cantilevers with a force constant of 0.2 Nm⁻¹ (Budget Sensors).

SEM studies were carried out on a Helios NanoLab G3 field-emission scanning electron microscope. Low-energy inverse photoemission spectroscopy (LEIPS, ALS Technology Co., Ltd) measurements were performed in a high-vacuum chamber with a pressure of 10-10 Torr, and the sample transfer was conducted with the aid of a custom-designed vacuum suitcase (Ferrovac GmbH), which ensures no ambient exposure during the transfer process.

X-ray diffraction measurements were carried out on a Bruker D8 Discover diffractometer (Bruker AXS) using a Cu (λ=1.54 angstrom) source with a power output of 1600 W.

The PL mapping profiles of large-area perovskite films were obtained using the Nanofinder (registered trademark) 30 system with an excitation wavelength of 480 nm. TAS measurements were conducted using a home-made pump-probe system constructed using CMOS detector (S12198-1024Q, Hamamatsu) and InGaAs photodiode array (G9203-256DA, Hamamatsu). An Yb: KGW laser (Light conversion, Pharos, 1030 nm, 1 kHz repetition rate, 150 fs, 2 mJ per pulse) was used as the main light source. The output from the laser was split into the pump beam and probe beam. The pump beam was directed into the optical parametric amplifier (Light conversion, ORPHEUS) to generate the 343 nm excitation and the probe beam was focused into a YAG plate to generate white light continuum. For mobility measurement, 663 VA Stand (Metrohm) system and PGSTAT128N (Metrohm) was employed to measure the J-V² plots of the ITO/hole-selective contact (thickness of 40 nm)/Hg. Liquid-state Hg electrode was used to form a soft contact at the metal/organic molecule interface for realizing better hole-injection process. The mobility could be derived from the Mott-Gurney equation, μh=8JL³/_{9ε0εr}V², where L is the thickness of hole-selective contact layer, co is the vacuum permittivity, and Er is the relative dielectric constant.

(In situ MS Characterization)

The in-situ MS measurement was conducted in a home-made vacuum chamber coupled with a solar simulator and temperature tracking system. The vacuum chamber with a background pressure of 10⁻⁸ Torr (CC-10, VISTA Corp.) is achieved by using a turbo molecular pump (TMP; HiCube80, Pfeiffer). On the top part of the chamber, a simulated sun-light is illuminated through the quartz window using a 150 W Xe lamp from the solar simulator (PEC-L01, Peccell Technologies, Inc.). The perovskite samples deposited on a 5 cm×5 cm substrates are fixed using a home-designed sample holder. A quadrupole mass spectrometer (HAL3F501RC, Hiden Analytical) with the m/z range of 1-510 amu and electron multiplier (to enhance signal detection) is mounted between the sample holder and TMP. Thermocouple (RX-450K, AsOne Corp.) was inserted into the vacuum chamber using electrical feedthrough and connected directly to the backside (glass side) of the samples allowing to obtain the real-time temperature evolution simultaneously with the MS measurements.

### (Operational Stability Measurements)

For the long-term operational stability measurements, the devices were loaded in a home-designed enclosure box under a flow of dry nitrogen to keep a relative humidity below 5% and a controllable temperature. Both small-sized cells and solar modules were continuously illuminated by a solar simulator (Peccell PEC-L01). Keithley 2401 source meter with the control of a customized Lab View program was used to automatically track the power output for a long time. In order to simulate operational conditions of a device, a fixed bias voltage close to the initial maximum power point (MPPT) voltage was applied to the devices during entire operational process. The power was recorded at a fixed time interval by the Lab View program according to the fixed bias and photocurrent output.

### (Theoretical Calculations)

The optimization of molecular geometry at the gas phase for MeO-2PACz, BPP, and SA-BPP were conducted on the Gaussian 09 program by using B3LYP with all-electron double-valence basis sets of 6-31G* based on the density functional theory (DFT) calculations. For the calculation of the absorption energy, In₂O₃ (100) slabs with the absorbed MeO-2PACz, BPP, and SA-BPP molecules were built, the lattice relaxation process was carried out in the framework of DFT method as implemented in the Vienna Ab Initio Simulation Package (VASP) program and about 20 angstrom vacuum space was introduced on the top of the model to reduce the interaction between adjacent slabs. The generalized gradient approximation in the form of Perdew-Burke-Ernzerhof was used for the exchange correlation function. Moreover, for the calculation of projected density of state FAPbI₃ (100) slabs with three layers of [PbI₆]⁴⁻ units and a 20 angstrom vacuum space were built on the top, and the bottom two layers of [PbI₆]⁴⁻ units were fixed during the geometry optimization process. The optimized BPP and SA-BPP molecules were added to the FAPbI₃ surface to simulate the passivation effect of surface under coordinated Pb²⁺ cations.

### (Synthesis: BPP—Br and SA-BPP)

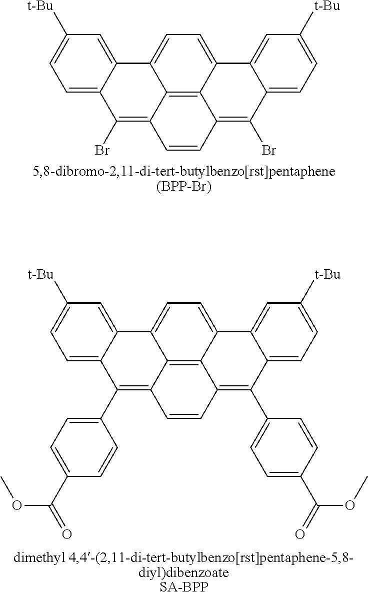

BPP—Br (A6) and SA-BPP were synthesized as shown in Scheme 1.

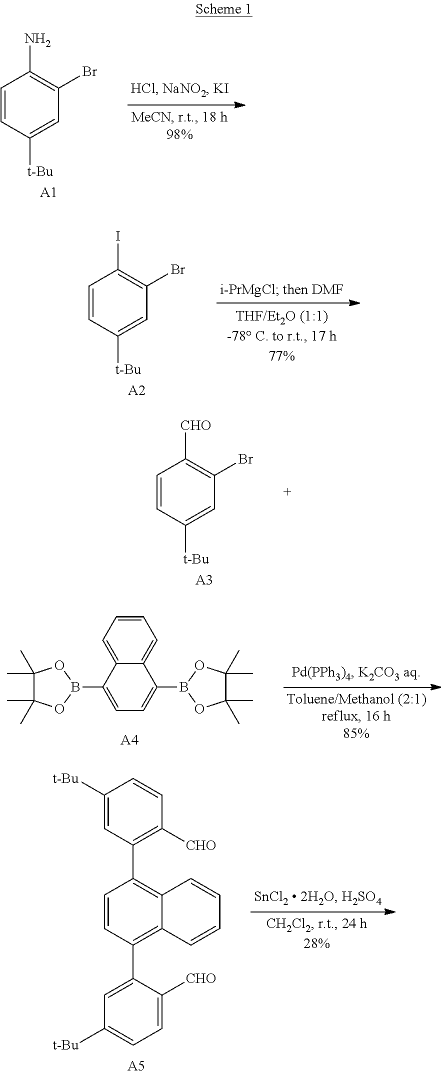

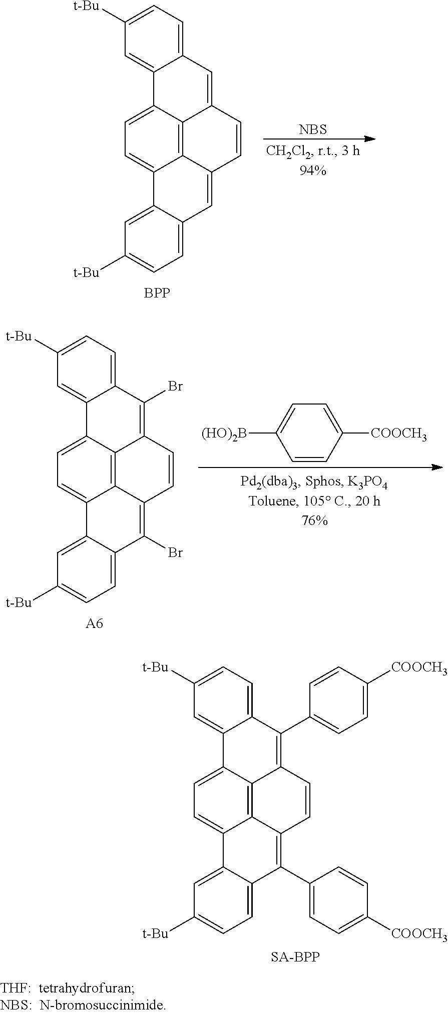

All reactions working with air- or moisture-sensitive compounds were carried out under argon atmosphere using standard Schlenk line techniques. Thin-layer chromatography (TLC) was done on silica gel coated aluminum sheets with F254 indicator and column chromatography separation was performed with silica gel (particle size 0.063-0.200 mm).

BPP and BPP—Br (A6) were synthesized as shown in Scheme 1. Briefly, 2-bromo-4-(tert-butyl)-1-iodobenzene (A2) was prepared by Sandmeyer reaction of 2-bromo-4-(tert-butyl) aniline (A1), followed by selective halogen-metal exchange to generate a corresponding Grignard reagent, which was then reacted with DMF to afford aldehyde (A3).

Suzuki-Miyaura coupling of aldehyde (A3) with diboronic ester (A4) gave dialdehyde (A5), which was subjected to “dehydrative π-extension” (DPEX) reaction to afford di-tert-butyl-benzo[rst]pentaphene (BPP).

Next, BPP was brominated by N-bromosuccinimide (NBS) to give BPP—Br (A6).

SA-BPP was finally obtained by Suzuki-Miyaura coupling of BPP—Br (A6) and 4-(methoxycarbonyl)phenylboronic acid in 76% yield.

The chemical structure of SA-BPP was unambiguously characterized by nuclear magnetic resonance (NMR) and high-resolution mass spectrometry (HRMS).

For the synthesis of dimethyl 4,4′-(2,11-di-tert-butylbenzo[rst]pentaphene-5,8-diyl)dibenzoate (SA-BPP), a 100-mL Schlenk tube was charged with 5,8-dibromo-2,11-di-tert-butylbenzo[rst]pentaphene (BPP—Br (A6)) (50.0 mg, 0.0877 mmol), 4-(methoxycarbonyl)phenylboronic acid (66.0 mg, 0.263 mmol), tris(dibenzylideneacetone)dipalladium(0) (Pd₂(dba)₃) (4.00 mg, 4.38 μmol), 2-dicyclohexylphosphino-2′,6′-dimethoxybiphenyl(SPhos) (3.60 mg, 8.77 μmol), K₃PO₄ (118 mg, 0.556 mmol), and anhydrous toluene (2 mL) under argon atmosphere. The reaction mixture was subjected to freeze-pump-thaw cycles (three times) and heated at 105° C. for 20 h under argon atmosphere. After the resulting mixture was cooled to room temperature and poured into water (20 mL), the organic layer was separated, and the aqueous layer was extracted with CH₂Cl₂ (10 mL) for three times. The separated organic phases were combined, washed with brine, dried over anhydrous MgSO₄, and evaporated. The residue was purified by silica gel column chromatography (eluent: hexane:EtOAc=10:1) to give SA-BPP (45.5 mg, 76% yield) as light yellow solid.

¹H NMR (400 MHz, CD₂Cl₂) δ 9.41 (s, 2H), 9.16 (d, J=1.9 Hz, 2H), 8.30-8.16 (m, 4H), 7.77 (dd, J=8.8, 1.8 Hz, 2H), 7.70 (dd, J=8.8, 0.5 Hz, 2H), 7.60-7.51 (m, 4H), 7.27 (s, 2H), 3.98 (s, 6H), 1.58 (s, 18H). ¹³C NMR (101 MHz, CD₂Cl₂) δ 166.85, 149.26, 144.24, 134.19, 131.49, 129.58, 128.71, 128.20, 126.97, 126.79, 126.14, 125.37, 124.49, 122.14, 118.42, 52.08, 35.33, 31.21. HRMS (ESI, Positive): 682.3078 [M]+, found: 682.3093.

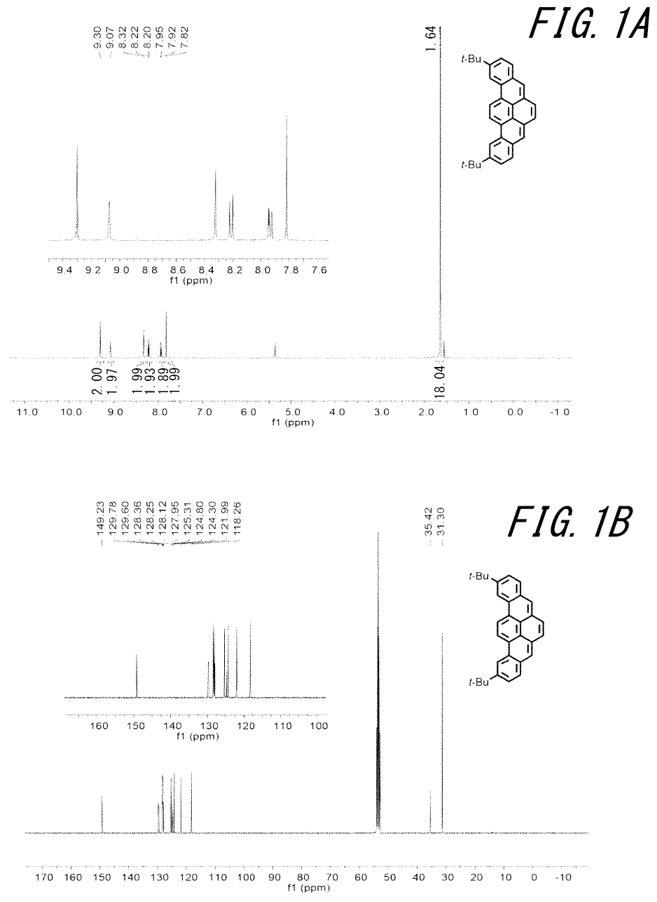

FIG. 1A shows ¹H NMR spectrum (400 MHz, 298 K) and FIG. 1B shows 13C NMR spectrum (101 MHz, 298 K) of BPP in CD₂Cl₂.

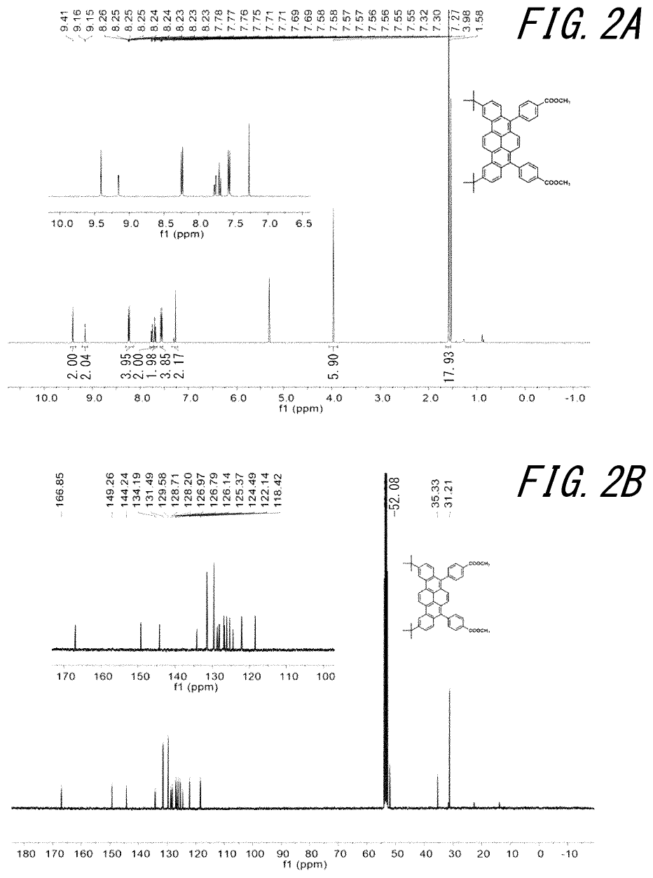

FIG. 2A shows ¹H NMR spectrum (400 MHz, 298 K) and FIG. 2B shows 13C NMR spectrum (101 MHz, 298 K) of SA-BPP in CD₂Cl₂.

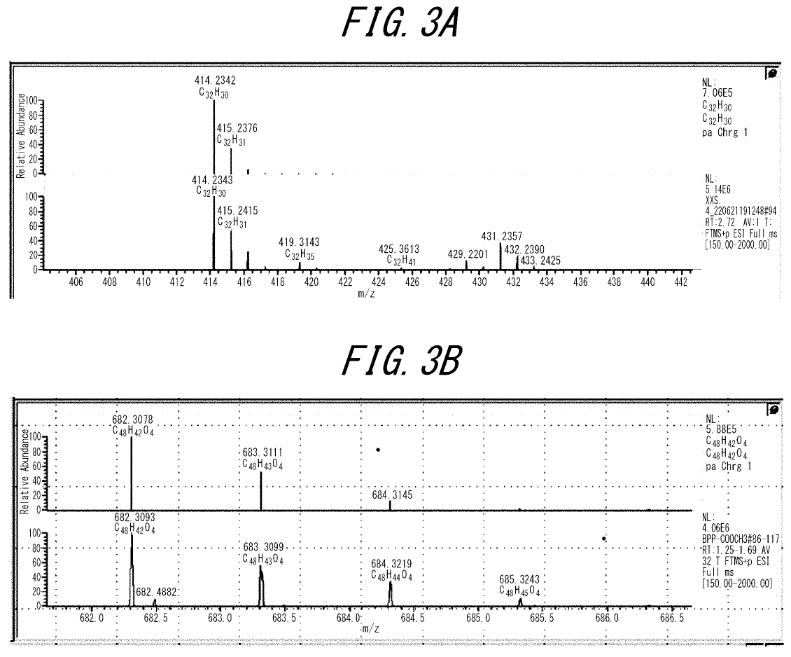

FIG. 3A shows high-resolution mass spectra of BPP and FIG. 2B shows SA-BPP (Top: calculated isotopic distribution pattern; down: experimental isotopic distribution pattern).

As shown in FIG. 2A and FIG. 2B, both BPP and SA-BPP show no detectable impurity peak in their NMR spectra, indicating high purity of the synthesized products. In particular, SA-BPP powder could be easily dissolved in the green solvents such as the commonly-used ethyl acetate thereby enabling the open-air fabrication of the SA-BPP hole-selective contact for the solar module production.

### (Synthesis: BPP-MeOPA)

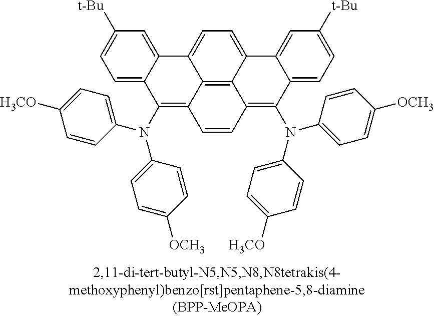 [SMILES: COc1ccc(N(c2ccc(OC)cc2)c2c3ccc(C(C)(C)C)cc3c3ccc4c5cc(C(C)(C)C)ccc5c(N(c5ccc(OC)cc5)c5ccc(OC)cc5)c5ccc2c3c54)cc1]

BPP-MeOPa was prepared synthesized by the method described in the following literature:

X. Xu et al., “Solvent-tunable exciton-charge transfer mixed state enhances emission of functionalized benzo[rst]pentaphene through symmetry breaking”, Chemical Communications, 2023, 2023,59, 720-723

### (Synthesis: BPP-Boc)

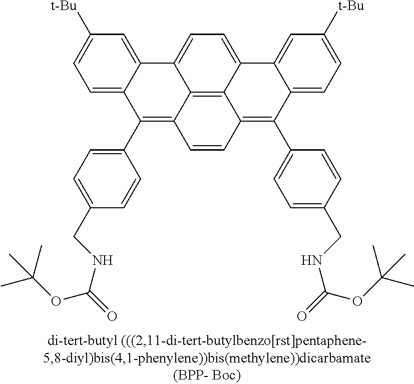 [SMILES: CC(C)(C)OC(=O)NCc1ccc(-c2c3ccc(C(C)(C)C)cc3c3ccc4c5cc(C(C)(C)C)ccc5c(-c5ccc(CNC(=O)OC(C)(C)C)cc5)c5ccc2c3c54)cc1]

BPP—BOC was synthesized as shown in Scheme 2.

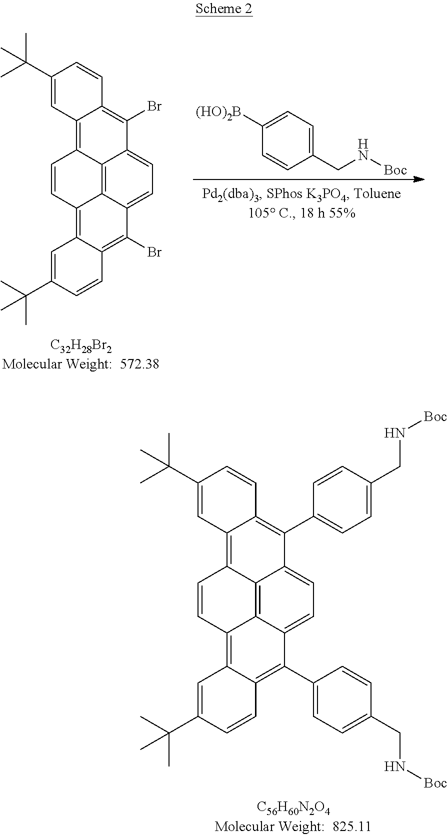 [SMILES: CC(C)(C)c1ccc2c(Br)c3ccc4c(Br)c5ccc(C(C)(C)C)cc5c5ccc(c2c1)c3c45 ; CC(C)(C)OC(=O)NCc1ccc(B(O)O)cc1 ; CC(C)(C)OC(=O)NCc1ccc(-c2c3ccc(C(C)(C)C)cc3c3ccc4c5cc(C(C)(C)C)ccc5c(-c5ccc(CNC(=O)OC(C)(C)C)cc5)c5ccc2c3c54)cc1]

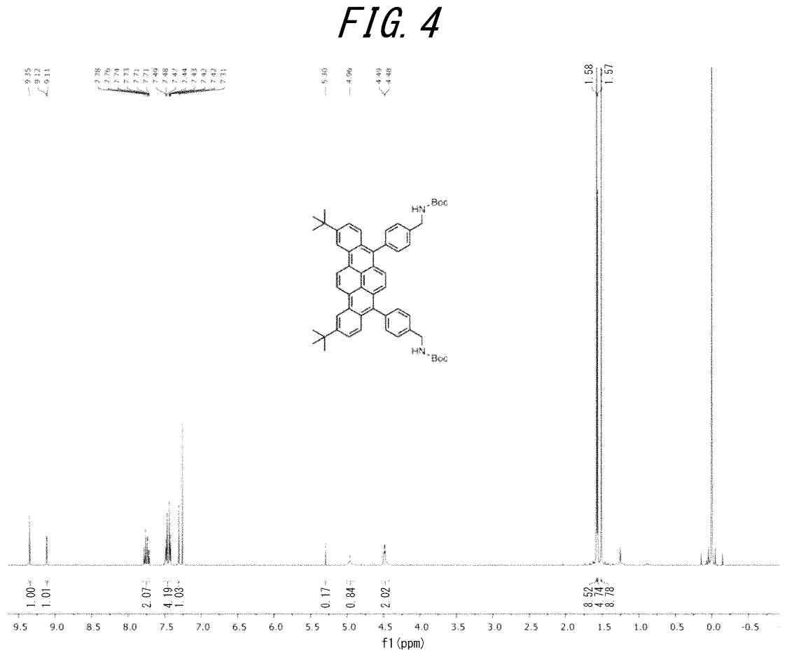

FIG. 4 shows ¹H NMR spectrum (400 MHz, 298 K) of BPP-Boc in CD₂Cl₂.

### (Density Functional Theory (DFT) Calculation)

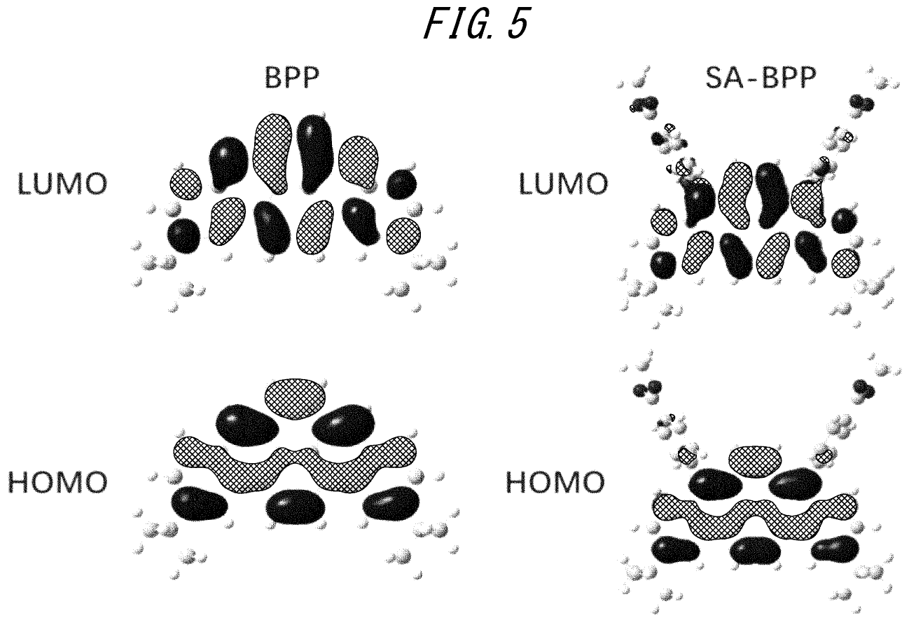

The density functional theory (DFT) calculation to reveal the distribution of the frontier molecular orbitals of BPP and SA-BPP. As can be seen in FIG. 5, both the highest occupied molecular orbital (HOMO) and the lowest unoccupied molecular orbital (LUMO) cover the fused six-membered benzenoid rings of BPP, indicating a strong delocalization of the electron cloud. In the case of SA-BPP, both the HOMO and LUMO are partially extended to the electron-withdrawing benzoate terminal groups. Such delocalized frontier orbital distribution can significantly improve the photostability of the organic semiconductors.

### (Properties of BPP and SA-BPP)

FIG. 6A shows chemical structures of the BPP and SA-BPP molecules and the schematic illumination of the SA-BPP hole-selective contact that could form the bonding with both ITO substrate and perovskite layer in inverted PSCs.

To reveal the band structure of these graphene-like molecules, the ultraviolet photoemission spectroscopy (UPS) and low-energy inverse photoemission spectroscopy (LEIPS) were performed to measure the HOMO and LUMO positions with respect to the Fermi level (EF). As demonstrated in FIG. 6C, the energy gaps between HOMO and LUMO were calculated to be 2.84 eV and 2.78 eV for BPP and SA-BPP, respectively, and the SA-BPP molecule shows a more p-type characteristic.

From the secondary electron cutoff feature of UPS spectra, the ionization potential of the BPP and SA-BPP was determined to be 5.05 eV and 5.24 eV, respectively, which suggests an energy offset of 0.41 eV and 0.22 eV with respect to the valence band maximum (VBM) of the perovskite film (cesium-formamidinium-methylammonium lead iodide (CsFAMAPbI₃) was used in this work). Such a deeper HOMO level of SA-BPP could be attributed to the decreased electron density on the BPP core caused by the electron-withdrawing benzoate terminal groups. Besides, the deeper EF position of SA-BPP compared to the BPP and pure ITO enables a barrier-free hole extraction process from perovskite absorber to the hole contact (FIG. 6D).

Then, the interaction between SA-BPP molecule and ITO or perovskite components was characterized by using Fourier transform infrared (FTIR) spectroscopy. As shown in FIG. 6B, the pure SA-BPP samples show the IR absorption features at 1726 cm⁻¹ (C═O stretching vibration), 1605 cm⁻¹ (C═C stretching vibration), 1438 cm⁻¹ (C—H bending vibration), 1282 cm⁻¹ (C—C stretching vibration), and 10⁹⁹ cm⁻¹ (Ph-H (the C—H bond on phenyl rings) bending vibration). It was found that the vibration peak intensity of C—O group decreased significantly with respect to the C═C (C—C) and C—H vibration peaks of SA-BPP after being mixed with ITO or PbI2 powders, indicating the formation of C═O . . . Pb bonding with the PbI2 or C—O . . . (Sn, In) bonding with the ITO that causes the electron cloud deformation of the C═O group.

### (Self-Anchoring Behaviors of BPP and SA-BPP)

The self-anchoring behaviors of BPP and SA-BPP molecules on the In₂O₃ (100) surface (simulating the ITO) were investigated through the DFT method. MeO-2PACz frequently used for fabricating the high-performance inverted PSCs was chosen as a reference sample. A strong coupling of the electron cloud between the phosphonic acid group and the surface In atom could be found in In₂O₃ (100)/MeO-2PACz sample, and the adsorption energy (Eads) was calculated to be −2.15 eV, but the BPP molecule showed a very small Eads of −0.34 eV on In₂O₃ (100) surface due to the lack of anchoring group. By contrast, the SA-BPP molecule/In₂O₃ (100) interface generated two obvious charge-redistribution sites owing to the bonding formed between the C—O groups and the In atoms on In₂O₃ surface, resulting in a much larger Eads of −2.61 eV.

These different anchoring behaviors were characterized by the XPS method. XPS C1s spectra of the MeO-2PACz, BPP, and SA-BPP films deposited on ITO glass before and after solvent washing was determined. The C Is peak intensity of MeO-2PACz film decreased to 88% and 76% of the initial value after being washed by isopropanol (IPA) and N,N-dimethylformamide (DMF), respectively, indicating the removal of excess surface nonbonding molecules. For BPP film, the C Is peak intensity drop to less than 30% of the initial value after being washed by DMF, which demonstrates the fact that BPP layer could be removed easily after the deposition of perovskite layer using solution-based method. On the contrary, the SA-BPP film kept over 80% of the initial peak intensity after being washed by DMF, which confirms the large bonding strength of SA-BPP on the ITO substrate.

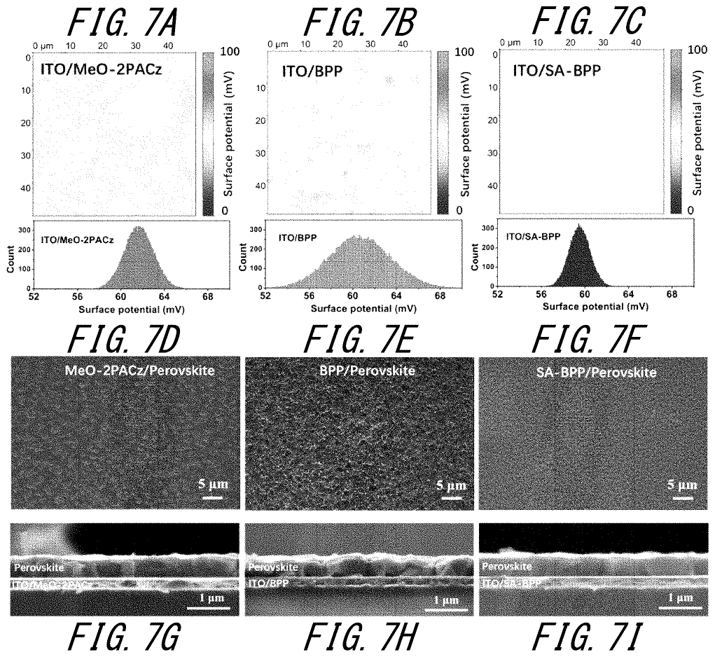

To visualize the uniformity of the hole-selective thin film coated on ITO glass, a surface-sensitive technique, scanning Kelvin probe microscopy (SKPM) was applied to achieve the surface potential mapping profiles of different hole-selective contacts on a 50 μm×50 μm region. As depicted in FIG. 7A to FIG. 7C, the BPP film shows a clear green (low potential) and red (high potential) regions in the mapping profile, while the SA-BPP film shows a uniform color mapping with a smaller difference between the minimum and the maximum potential values than that of the BPP and MeO-2PACz films. A possible explanation is that the double-site anchoring effect of the SA-BPP molecule leads to a more ordered loading on the ITO surface. On the contrary, the heterogeneous potential distribution of BPP film could be attributed to the intermolecular aggregation induced by strong π-π interaction.

Scanning electron microscope (SEM) measurement was conducted to study the morphology of perovskite films deposited on different hole-selective contacts. As shown in FIG. 7D to FIG. 7F, the perovskite films deposited on MeO-2PACz and SA-BPP exhibited a smoother surface morphology compared with the perovskite film grown on BPP.

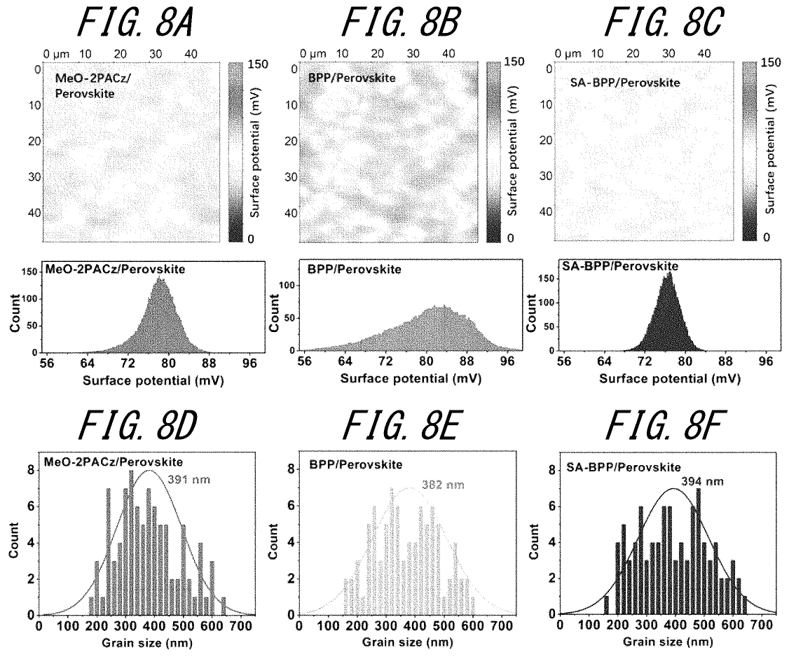

Also, surface potential mapping profiles (50 μm×50 μm) of the perovskite films deposited on SA-BPP showed a much lower potential fluctuation than that of the perovskite films coated on BPP and MeO-2PACz (FIG. 8A to FIG. 8F). On the other hand, cross-section SEM images (FIG. 7G to FIG. 7I) indicate a pinhole-free contact for the ITO/MeO-2PACz/perovskite and ITO/SA-BPP/perovskite samples, while a random orientation of the perovskite grains with many pinholes was found at the BPP/perovskite interface, which usually leads to a non-radiative recombination of the charge carriers.

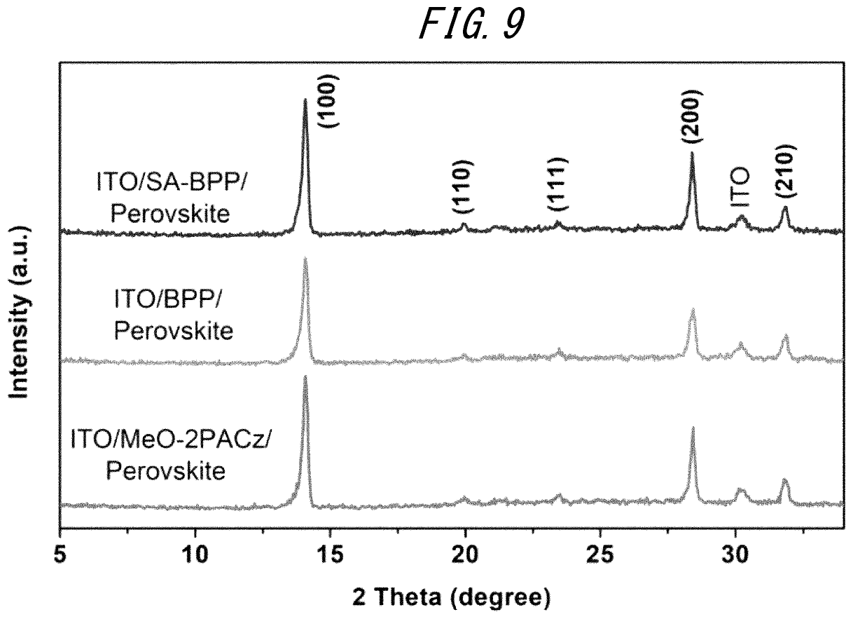

Next, the crystal structures of perovskite films were characterized by X-ray diffraction (XRD) measurement. FIG. 9 depicts the XRD patterns of different samples, the perovskite film on BPP substrate showed a weaker peak intensity of the (100) and (200) crystal planes that are crucial for the charge carrier transport in perovskite absorber compared to the XRD patterns of SA-BPP/perovskite or MeO-2PACz/perovskite samples. The above results illustrate that the strong chemical bonding between hole-selective contact and perovskite buried interface is relevant and believed to be crucial for the growth of compact and highly oriented perovskite crystals.

### Example 2 (Interface Charge Carrier Dynamics)

(TAS measurements)

The interface carrier dynamics through femtosecond transient absorption spectroscopy (TAS) technique were performed. Sample structures of glass/hole-selective contacts/perovskite layer were employed for TAS measurements.

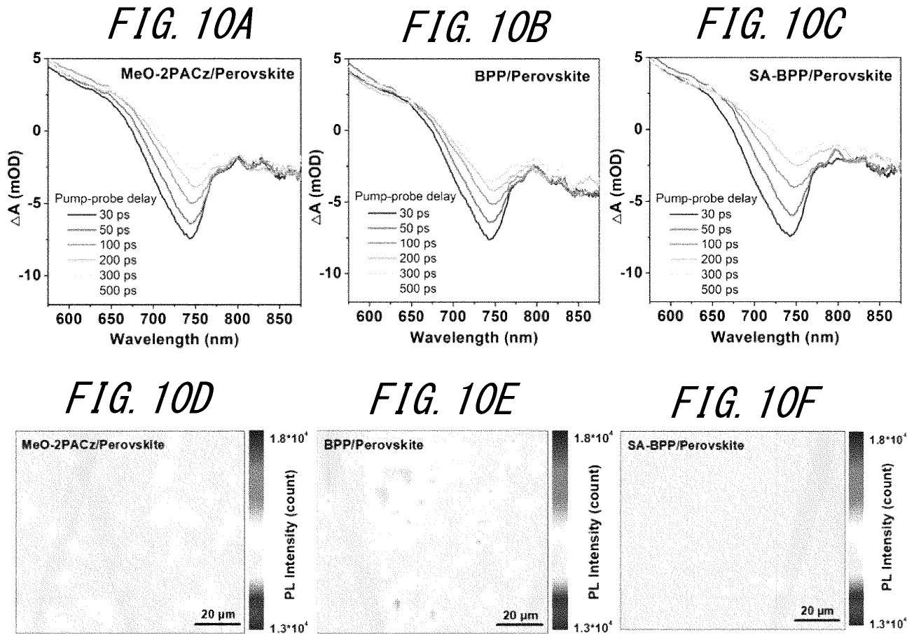

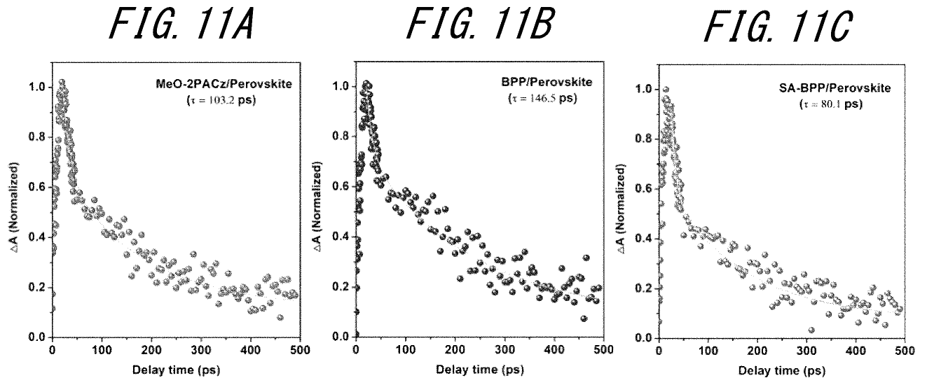

As shown in FIG. 10A to FIG. 10C, a unique ground-state bleaching (GSB) peak of the CsFAMAPbI₃ perovskite was formed at around 749 nm, and the quenching process of the GSB peak was detected at the pump-probe delay time from 30 ps to 500 ps. The quenching rate of GSB peak signal indicates the carrier transport efficiency from perovskite absorber to the hole contact. A larger quenching of over 95% of the GSB peak intensity was found in SA-BPP/perovskite film from the delay time of 30 ps to 500 ps compared to that of the MeO-2PACz/perovskite (81%) and BPP/perovskite (64%) films, reflecting a high hole extraction efficiency at the SA-BPP/perovskite interface. Then, the decay lifetimes of GSB peak to be 103.2 ps, 146.5 ps, and 80.1 ps for the MeO-2PACz/perovskite, BPP/perovskite, and SA-BPP/perovskite were calculated, respectively, by fitting the decay kinetics plots of TAS spectra (FIG. 11A to FIG. 11C).

### (PL Mapping)

Moreover, the uniformity of the charge extraction process at the hole contact interface was investigated through photoluminescence (PL) mapping techniques. FIG. 10D to FIG. 10F show the PL mapping profiles of the corresponding samples on a 100 μm×100 μm region, the blue color region indicates a strong PL quenching effect while the red color region indicates a weak PL quenching effect. The MeO-2PACz/perovskite and BPP/perovskite samples show a heterogeneous PL mapping intensity, reflecting a large difference of the charge extraction behaviors. Such differences in PL characteristics at the micro-scale region could be ascribed to the heterogeneity of perovskite morphology and hole contact quality. By contrast, the PL mapping profile of SA-BPP/perovskite demonstrates a more uniform hole extraction process, which is relevant and believed to be crucial for realizing an efficient charge carrier separation in the large-area device.

### (Other Measurements)

Generally, the charge extraction process is highly affected by the carrier (uh). The hole mobility (uh) of different hole-selective contacts was estimated by calculating the slope of the dark current density-voltage² (J-V²) plot according to the Mott-Gurney equation. Here, the hole-only devices containing MeO-2PACz or SA-BPP layer were employed for measuring the J-V² plots. The uh of SA-BPP is calculated to be (4.27±0.29)×10⁻⁴ cm² V⁻¹ s⁻¹, which is of 6 times higher than that of MeO-2PACz (μh=(7.12±0.15)×10⁻⁵ cm² V⁻¹ s⁻¹).

In addition to mobility, the trap density (Ntrap) at the perovskite buried interface should also be an important factor in determining hole extraction efficiency. In this work, the Ntrap value was measured by the space charge limit current (SCLC) method.

The Ntrap values of the MeO-2PACz, BPP, and SA-BPP-based samples were estimated to be 7.95×10¹⁵ cm⁻³, 9.93×10¹⁵ cm⁻³, and 6.34×10¹⁵ cm⁻³, respectively, indicating the fact that SA-BPP is able to minimize the trap states and thus enables a fast extraction of the photogenerated hole in perovskite absorber.

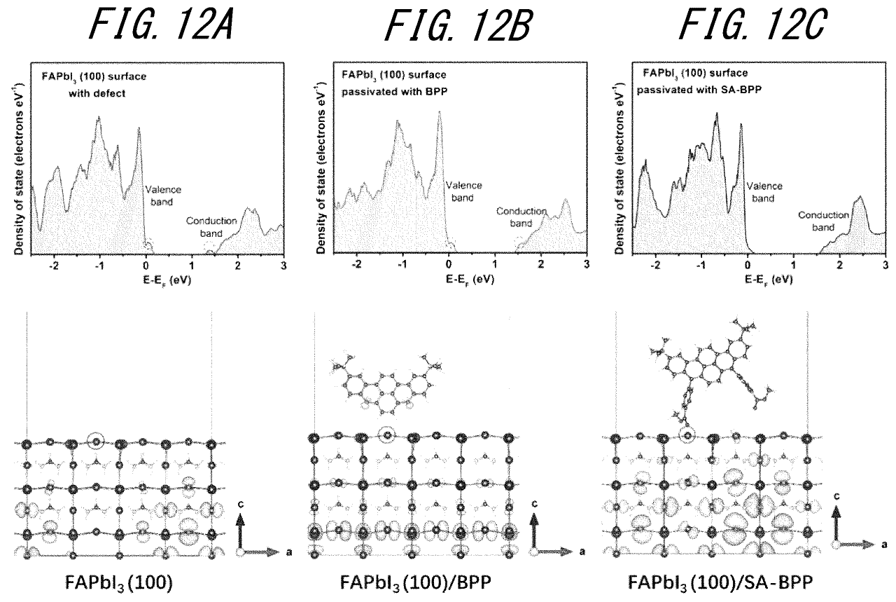

The passivation mechanism of the SA-BPP contact has been uncovered via simulating the interaction between SA-BPP or BPP molecules and the iodide vacancy that has been regarded as the main source of defects for iodide-based perovskite materials, and a pure cubic FAPbI₃ perovskite model was applied here to simplify the calculation process. FIG. 12A to FIG. 12C illustrate the density of state (DOS) distribution of the FAPbI₃ (100) slabs without and with the passivation of SA-BPP or BPP, and the insets show the corresponding geometry-optimized structures with an iodide vacancy on the surface (indicated by blue circle). The pristine FAPbI₃ slab shows a localized charge distribution of the HOMO orbital, which generates additional trap states at both valence and conduction band edges, resulting in recombination of the electrons and holes. These calculation results are consistent with the previous ab initio analysis of the defect-containing perovskite structure. In the case of BPP-passivated FAPbI₃ slab, the trap states still presented at band edges owing to the weak electronic coupling between the BPP core and the surface under-coordinated Pb cations. In contrast, the chemical bonding between the C—O groups of SA-BPP and surface under-coordinated Pb cations effectively delocalizes the charge distribution around the Pb—I frameworks, which minimizes the trap density at the valence and conduction band edges in SA-BPP-passivated FAPbI₃ model.

### Example 3 (Stability of Hole-Selective Contact and Relative Interface)

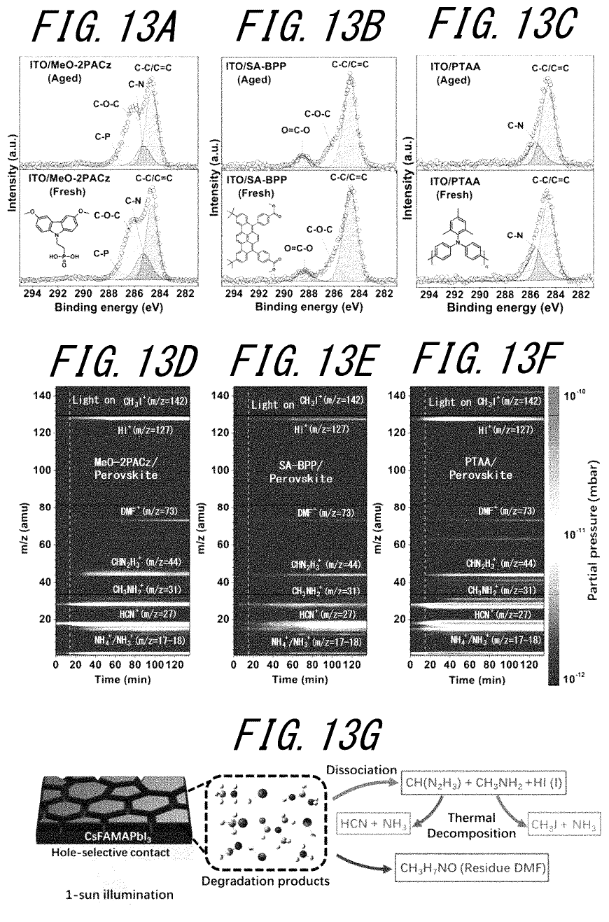

For a systematical comparison, the stability of SA-BPP hole-selective contact under continuous 1-sun illumination was further investigated, and the frequently-used carbazole-based MeO-2PACz and triphenylamine-based PTAA were used as reference samples. The evolution of their XPS C Is core levels before and after the aging process can be seen in FIG. 13A, the separated peaks at 284.5 eV, 285.3 eV, 286.2 eV, and 287.3 eV are associated with the C—C(C═C), C—N, C—O—C, and C—P bonds of the MeO-2PACz, respectively. It was found that the intensity of C—N and C—P peaks decreased significantly after aged at 1-sun illumination for 100 hours, indicating a degradation of the carbazole core and the phosphonic acid anchoring group. In contrast, no obvious change of the C—C, C—O—C, and O═C—O peak intensities for SA-BPP sample (FIG. 13B) was found, reflecting that the BPP core and the benzoate terminal group kept stable after aged at the same condition. The PTAA contact also showed a severe light-induced degradation of the triphenylamine unit with about 30% decay of the peak intensity of C—N bond after the aging process (FIG. 13C).

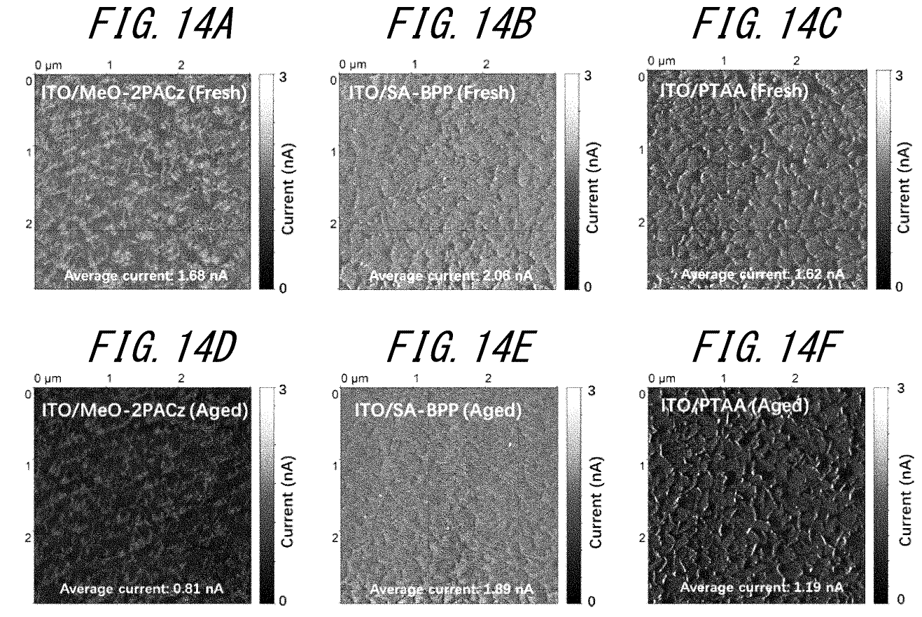

Further, the conductive atomic force microscope (C-AFM) measurement was performed to study the impact of structure evolutions on the conductivity of these hole-selective contact. As shown in FIG. 14A to FIG. 14C, the C-AFM current mapping profiles of MeO-2PACz film show a significant drop of the average current from 1.68 nA to 0.81 nA after aged at 1-sun illumination for 100 hours under the same applied bias, and the mapping current of the PTAA film showed a similar decay trend (from 1.62 nA to 1.19 nA). By contrast, a negligible change of the mapping current (0.17 nA) could be found in the SA-BPP film after aging process. The above results highlight the excellent photostability of the SA-BPP with respect to the MeO-2PACz and PTAA benchmarks.

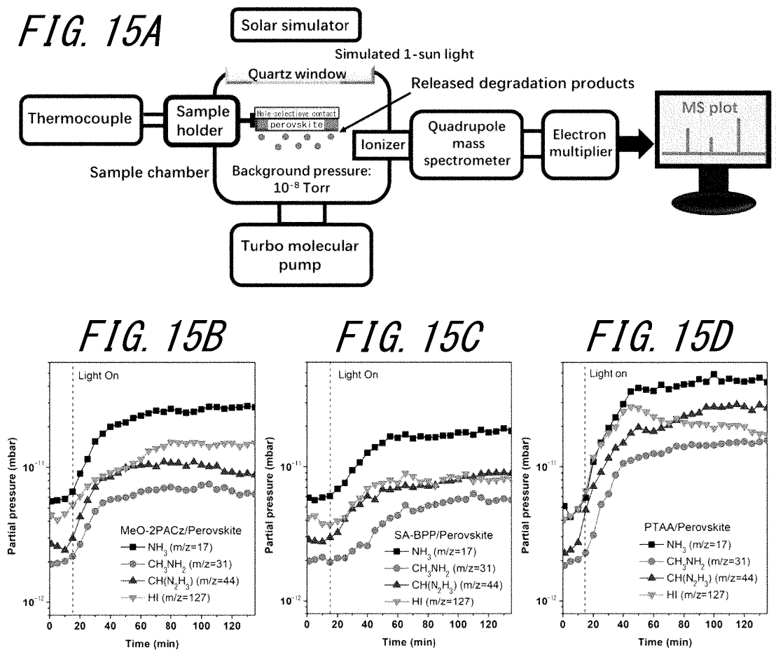

In addition to the hole-selective contact itself, the relative perovskite buried interface also plays a critical role on the long-term operational stability of inverted PSCs. In situ mass spectrometry (MS) technique is a power tool for the stability study, which can identify the gas-phase degradation products and reveal their partial pressure evolution under the influence of light and heat. This technique was applied in our previous studies for revealing the temperature-dependent or light-dependent degradation mechanisms of the perovskite materials and relative interfaces. Here an MS system was employed to track the released degradation products from the hole-selective contact/perovskite interface under simulated 1-sun illumination in a vacuum chamber with a background pressure of 10⁻⁸ Torr. The schematic diagram of this in-situ MS system is illustrated in FIG. 15A. FIG. 13D to FIG. 13F show the MS contour plots with the mass-to-charge ratio (m/z) of 1-200 amu for the perovskite films deposited on MeO-2PACz, SA-BPP, and PTAA substrates, and the possible degradation pathways derived from the MS plots were summarized in FIG. 13G. The degradation products with a m/z of 17, 27, 31, 44, 73, 127/128, 142 amu were detected after exposed to 1-sun light, associated with the NH³⁺, HCN⁺, CH₃NH²⁺ (MA gas), CH(N₂H₃)⁺ (FA gas), DMF⁺ (N,N-dimethylformamide solvent), I⁺/HI⁺ (hydrogen iodide), and CH₃I⁺ (methyliodide), respectively. Also, the fragments induced by the ionization process of mass analysis were detected, including CH³⁺ (m/z=15, methyl group), NH²⁺ (m/z=16, amino group), CH₄N⁺ (m/z=30, MA fragment), CH₂N₂⁺ (m/z=42, FA fragment), and I₂⁺ (m/z=63, iodide fragment). The generation of gas-phase FA, MA, and HI could be attributed to the deprotonation reaction of organic ammonium components. The deprotonation process can be accelerated under continuous illumination or elevated temperature, as illustrated by the increased partial pressure of the corresponding components. Differently, the formation of NH₃, HCN, and CH₃I were induced by the thermal decomposition of CH(N₂H₃) and the reaction between CH₃NH₂ and HI. As shown in FIG. 15B to FIG. 15D, in the case of MeO-2PACz/perovskite, the partial pressure of NH₃, CH₃NH₂, CH(N₂H₃), and HI increased by 3-4 times after 120 min of illumination, and the partial pressure of CH₃NH₂ and CH(N₂H₃) increased by about one order of magnitude for the PTAA/perovskite sample. By contrast, less degradation products were released from the SA-BPP/perovskite sample, which means that the deprotonation and decomposition of the organic components in perovskite absorber could be effectively suppressed under operational condition, which could be attributed to the good perovskite crystal quality induced by the SA-BPP contact as well as the enhanced interface stability.

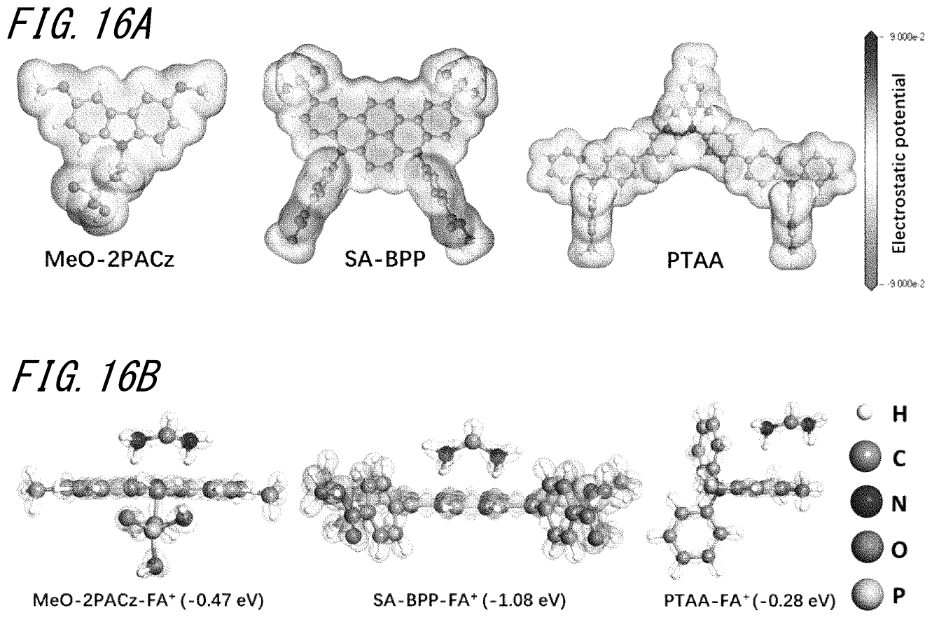

To further illustrate the possible reason for the stability improvement at perovskite buried interface, the electrostatic potential (ESP) profiles of MeO-2PACz, SA-BPP, and PTAA were calculated based on the DFT method (FIG. 16A). For the MeO-2PACz and PTAA models, negative ESP (indicated by red color) mainly locates at the electron-rich carbazole unit and the benzene rings of triphenylamine unit, while a much higher electron density at the BPP core of SA-BPP could be found. The high electron density at BPP core could be attributed to the strong delocalized effect of the π electrons, and is able to provide the active sites for forming cation-π interactions with the FA+ or MA⁺ cations. As demonstrated in FIG. 16B, the interaction energy between SA-BPP molecule and a FA+ cation is calculated to be −1.08 eV, much higher than that of the MeO-2PACz-FA⁺ (−0.47 eV) or PTAA-FA⁺ (−0.28 eV) complexes. Such cation-π interaction is able to fix the volatile organic cations at the perovskite buried interface and thus suppress the light-induced degradation of perovskite crystal under operational condition.

### Example 4 (Device Performance and Stability)

### (Fabrication of Small-Sized PSCs)

The ITO glass substrate was sequentially washed with distilled water, isopropanol, and acetone. After being dried by N₂ flow, the substrate was treated by the ultraviolet light (UV)/O₃ for 40 min. Afterwards, the hole-selective contact layers were deposited on the ITO substrate by spin-coating 0.003 M precursor solutions (ethyl alcohol for MeO-2PACz, ethyl acetate for SA-BPP, and chlorobenzene for BPP) at 3000 r.p.m. for 30 s, and then annealed at 100° C. for 15 min. For the deposition of PTAA layer on ITO substrate, 1 mg mL⁻¹ PTAA toluene solution was spin-coated onto the substrate at 5000 r.p.m. for 30 s, and then annealed at 120° C. for 20 min. Subsequently, a solution with 1.35 M PbI₂ and 0.0675 M CsI dissolved in DMF/DMSO (19:1, v/v) was spin-coated on the substrate at 3000 r.p.m. for 30 s in the N₂ glove box. Next, a mixed organic cation solution (60 mg FAI, 20 mg MAI, and 5 mg MACI dissolved in 1 ml isopropanol) was spin-coated at 3000 r.p.m. for 30 s and then annealed at 150° C. for 10 min in air, which formed a triple-cation Cs_{0.05}(FA_{0.78}MA_{0.22})_{0.95}PbI₃ perovskite layer according to our previous study. After that, 5 mg ml⁻¹ PCBM chlorobenzene solution was spin-coated on the perovskite surface at 1000 r.p.m. for 30 s and annealed at 70° C. for 10 min. Finally, 20 nm C₆₀, 5 nm BCP, and 80 nm Ag electrode were evaporated onto the substrate sequentially under a vacuum condition of 10⁻⁷ Torr.

The small-sized PSCs using BPP—Br, BPP-MeOPA and BPP-Boc were prepared in similar manner mentioned above.

### (Fabrication of Perovskite Solar Modules)

The perovskite solar module consisted of seven perovskite subcells connected in series on a 5 cm×5 cm ITO substrate. There P1 pattern of ITO substrate was designed by the Yingkou OPV Tech New Energy. The ITO substrate with P1 pattern was cleaned and then deposited with a hole-selective contact layer by using the spin-coating method described above. The perovskite layers were spin-coated onto the 5 cm×5 cm substrates based on the same sequential deposition method for the small-sized PSCs, followed by a two-step annealing process at 90° C. and 150° C. for 1 min and 10 min, respectively. After the deposition of PCBM and C60 layers, the samples were etched by a picosecond laser (Picosecond Laser Processing System, LPS-R002A, Spectronix Corporation), a 532-nm wavelength laser (power of 3.0 W) with a pulse duration of 12 ps is applied to create the P2 pattern. Subsequently, 5 nm of compact tin oxide layer was deposited on the C60 surface via an atomic layer deposition system (ALD, Cambridge Nanotechnology, Savannah S200) at low temperature, tetrakis(dimethylamino) tin (IV) (TDMASn) was chosen as the tin source and the growth rate of the ALD-SnO₂ layer was fixed at 0.53 angstrom per cycle. Afterwards, the samples were transferred into a high-vacuum chamber and 80 nm Ag electrode was evaporated onto the ALD-SnO₂ protection layer. Finally, a laser with a power of 3.8 W was used to form the P3 pattern after evaporation of Ag.

### (Characterization of PSC Performance)

The device J-V curves were measured under AM1.5G light illumination (100 mW cm⁻², calibrated by a KG3 reference silicon cell (Enlitech)) using a solar simulator (Newport Oriel Sol 1A, xenon lamp, USHIO, UXL-150SO) and Keithley 2420 source meter without preconditioning at room temperature and a relative humidity of 30%. For the small-sized cells, the J-V scan range was from −0.1 to 1.2 V and a metal mask with an aperture area of 0.1 cm² was used for the measurement. For the 5 cm×5 cm solar modules, the J-V scan range was from −0.1 to 8.5 V and a mask with an aperture area of 22.4 cm² was used for the measurement. The IPCE spectra of the inverted PSCs were characterized using Oriel IQE 200 system.

### (Photovoltaic Performance)

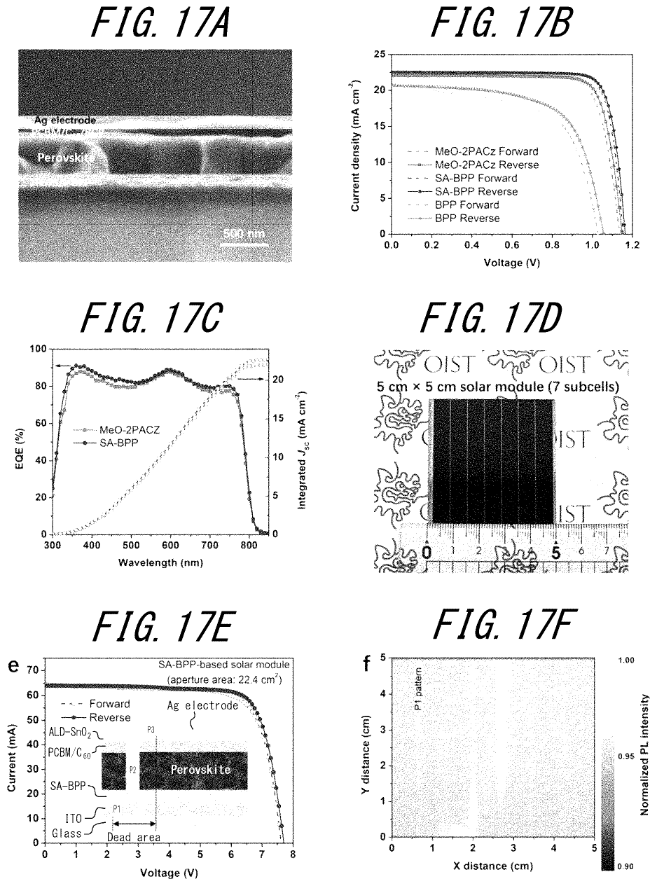

The inverted planar PSCs with a structure of ITO/SA-BPP/perovskite (500-550 nm)/PCBM/C₆₀/BCP/Ag (FIG. 17A) were fabricated to study the impact of SA-BPP on the photovoltaic performance, the MeO-2PACz and BPP being used as the reference hole-selective contacts for a comprehensive comparison. FIG. 17B plots the J-V curves of the small-sized PSCs with an aperture area of 0.1 cm² based on different hole-selective contacts, and the photovoltaic parameters of the champion devices were summarized in Table 1. The MeO-2PACz devices provide the best PCE of 20.57% (reverse scan), with an open-circuit voltage (VOC) of 1.15 V, short-circuit current density (JSC) of 22.11 mA cm 2, and fill factor (FF) of 80.89%. By contrast, SA-BPP devices show an obvious improvement on the photovoltaic performance and present the best PCE up to 22.03%, with a VOC of 1.17 V, JSC of 22.55 mA cm 2, and FF of 83.50%. On the contrary, a relatively low PCE of 14.51% was obtained by the BPP devices, with a VOC of 1.06 V, JSC of 20.65 mA cm 2, and FF of 66.31%. In this regard, we attributed the efficiency improvement of SA-BPP devices to the fast hole extraction process, efficient passivation of the perovskite buried interface, and the high mobility of the large-conjugation SA-BPP molecule. Also, the reproducibility of the SA-BPP devices is higher than the reference samples in view of the narrower statistical distribution of the photovoltaic parameters.

### (External Quantum Efficiency (EQE) Spectra)

As depicted in FIG. 17C, the external quantum efficiency (EQE) spectra of the corresponding PSCs were investigated. It was found that the EQE at the short wavelength region of 350-480 nm increased significantly when a SA-BPP hole-selective contact was incorporated. This EQE enhancement was associated with the improvement in carrier extraction efficiency at the illumination side (perovskite buried interface), which further supports the results from TAS and PL measurements. The integrated JSC derived from EQE spectra were calculated to be 22.08 mA cm⁻² and 22.49 mA cm⁻² for the MeO-2PACz and SA-BPP devices, respectively, consistent with the JSC derived from J-V measurements.

TABLE 1
The best photovoltaic performance of the small-sized cells and
solar modules based on different hole-selective contacts:

| | Scan | J_{SC} | V_{OC} | FF | PCE |
|---|---|---|---|---|---|
| Hole-selective contact | direction | [mA cm⁻²] | [V] | [%] | [%] |
| MeO-2PACz | forward | 22.14 | 1.14 | 79.78 | 20.14 |
| | reverse | 22.11 | 1.15 | 80.89 | 20.57 |
| SA-BPP | forward | 22.57 | 1.16 | 82.09 | 21.49 |
| | reverse | 22.55 | 1.17 | 83.50 | 22.03 |
| BPP | forward | 20.71 | 1.03 | 63.18 | 13.48 |
| | reverse | 20.65 | 1.06 | 66.31 | 14.51 |
| SA-BPP | forward | 2.88 | 7.59 | 75.06 | 16.41 |
| (22.4 cm² solar module) | reverse | 2.87 | 7.68 | 77.51 | 17.08 |

### (Evaluation of Solar Module)

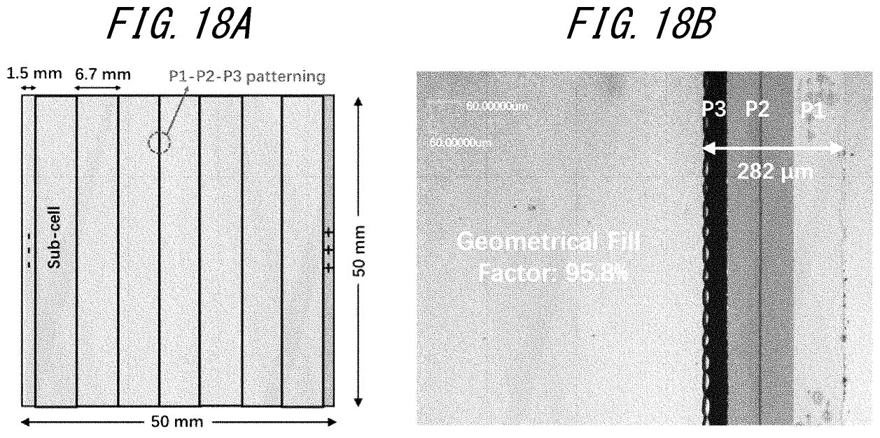

To evaluate the potential of SA-BPP for the large-scale production of inverted PSCs, the 5 cm×5 cm solar module consisted of 7 perovskite subcells connected in series was prepared (FIG. 17D). The deposition methods of different functional layers are similar to that of the small-sized cells, including the SA-BPP contact (spin-coating), perovskite absorbers (spin-coating), electron-selective contact (evaporation), and Ag electrode (evaporation). The architecture of solar module is illustrated in FIG. 17E, and the corresponding pattern design is shown in FIG. 18A. The geometrical fill factor (GFF) was calculated to be 95.8% according to the width of subcell and the dead area between P1 and P3 (FIG. 18B). Additionally, a conformal and compact tin oxide layer was deposited atop the P2 pattern via atomic layer deposition (ALD) technique. This ALD-SnO₂ layer was applied to prevent the direct contact between iodide-based perovskite absorber and Ag electrode that is able to accelerate the degradation of inverted PSC module under light and heat conditions.

For SA-BPP-based solar module, the corresponding J-V curves (FIG. 17E) show a reverse-scan PCE of 17.08% on an aperture area of 22.4 cm² (active area PCE of 17.83%), with a VOC of 7.68 V, JSC of 2.87 mA cm 2, and FF of 77.51%. Regarding the MeO-2PACz-based solar module, a relatively low PCE of 14.77% was obtained (active area PCE of 15.42%), with a VOC of 7.48 V, JSC of 2.81 mA cm 2, and FF of 70.28%. Large-area PL mapping technique was also employed to check the uniformity of the perovskite films deposited on 5 cm×5 cm substrate. As can be seen in FIG. 17F, the perovskite film coated on 5 cm×5 cm ITO/SA-BPP substrate enabled a more uniform PL mapping intensity than that of the film coated on ITO/MeO-2PACz substrate, the bright lines of mapping profiles indicate a relatively weak PL quenching effect at the P1 pattern of ITO glass.

### (Operational Stability)

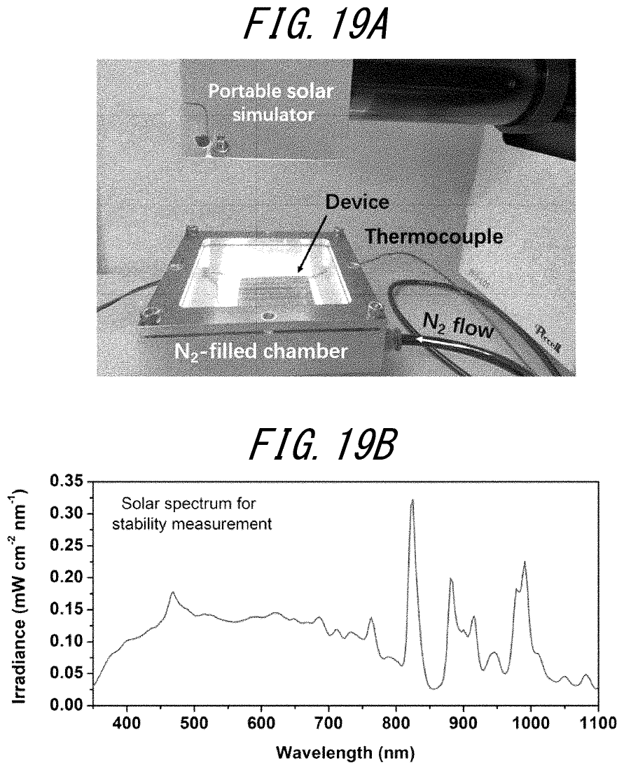

The operational stability of the PSC small-sized cells and modules under accelerated-ageing conditions based on the International Summit on Organic Photovoltaic Stability (ISOS) protocols were evaluated, where a home-designed test system was used for stability measurement (FIG. 19A), and a xenon white light source was employed to simulate the solar irradiation (the spectrum is shown in FIG. 19B).

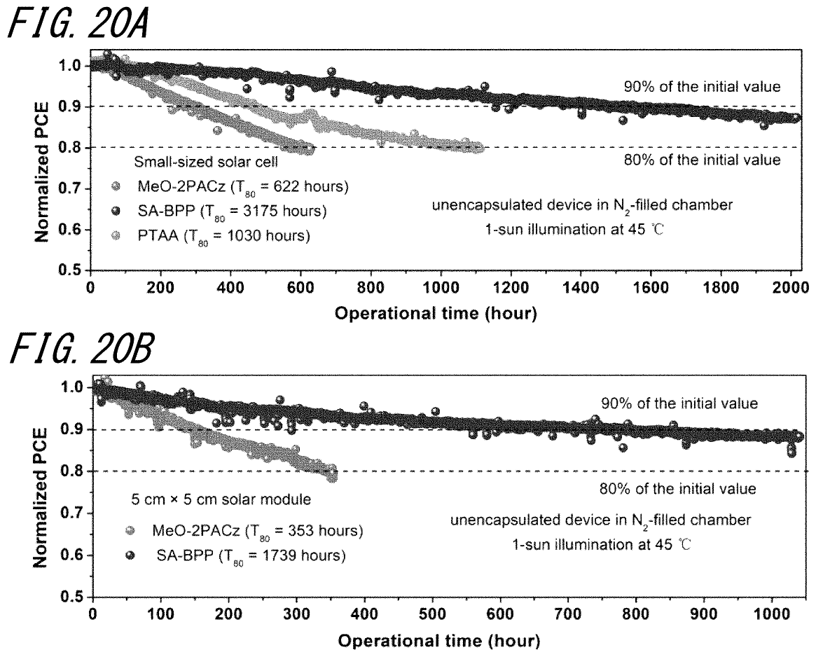

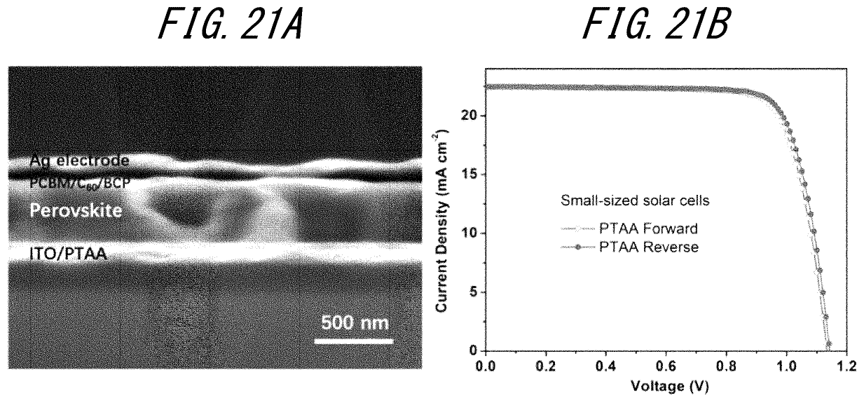

FIG. 20A demonstrated the normalized PCE decay curves of the unencapsulated PSCs based on MeO-2PACz, SA-BPP, and PTAA hole-selective contacts measured under 1-sun illumination at 45° C. (ISOS-L-21), and the decay plots with actual PCE values were presented in FIG. 21A and FIG. 21B. The reference MeO-2PACz and PTAA devices showed a normalized PCE decrease of over 20% after continuous operation for 622 hours and 1,030 hours, respectively. By contrast, the SA-BPP devices showed a linear-shape PCE decay plot and kept about 87.4% of the initial efficiency after 2,000-hour operation, which translates into a T80 lifetime of 3,175 hours that is among the highest operational lifetimes for the inverted PSCs.

TABLE 2
Summary of the operational stability under
simulated 1-sun illumination (100 mW cm⁻²)

| Hole- | Reported | Operation | Normalized | Average |
|---|---|---|---|---|
| Selective | PCE | time | PCE loss | PCE decay rate |
| Contact | [%] | [hour] | [%] | [% hour⁻¹] |
| SA-BPP | 22.0 | 2000 | (Unencapsulated | 0.0063 |
| | | | device in N₂ at | |
| | | | 45° C.) 20.4 | |
| MeO- | 20.6 | 630 | (Unencapsulated | 0.0324 |
| 2PACz | | | device in N₂ at | |
| | | | 45° C.) 20.2 | |
| PTAA | 20.2 | 1050 | (Unencapsulated | 0.0192 |
| | | | device in N₂ at | |
| | | | 45° C.) | |

The stability reproducibility of the SA-BPP devices was evaluated by tracking the PCE decay profiles of 6 individual cells under the same condition (1-sun illumination with dry N₂ flow). All of the devices exhibited a similar PCE decay rate between 0.0037% hour⁻¹ and 0.0041% hour⁻¹ in an operational time of 500 hours.

The operational stability of solar modules is one of the most important issues for the commercialization of PSCs. In this regard, the PCE decay of 5 cm×5 cm inverted PSC modules versus the operational time was tracked as shown in FIG. 20B (ISOS-L-2I). The SA-BPP-based module retained 88.5% of the initial efficiency after operation for 1,000 hours, which enables a much longer estimated T80 lifetime of 1,739 hours compared to that of the MeO-2PACz-based module (353 hours).

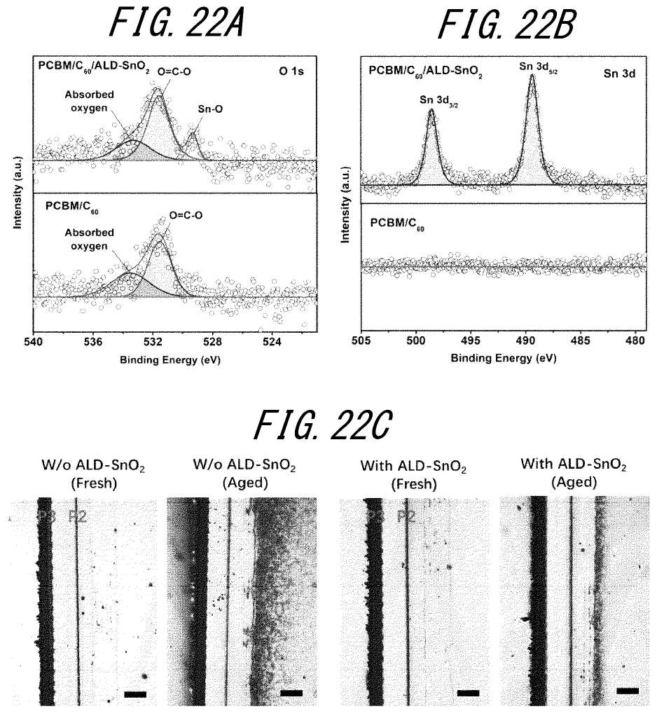

A solar modules (P2 and P3 patterns) where a compact ALD-SnO₂ layer was deposited to suppress the reaction between Ag electrode and the iodide components of perovskite layer induced by the lateral ion diffusion at the connection area. The XPS high-resolution O 1s (FIG. 22A) and Sn 3d (FIG. 22B) spectra indicate the formation of tin oxide on the electron-selective contact (PCBM/C60) surface. From the optical microscope images (FIG. 22C), we found that the corrosion of Ag electrode at the edge of P2 and P3 under 1-sun illumination could be retarded when this ALD protection layer was applied.

### (Evaluation of PSCs Using BPP—Br, BPP-MeOPA and BPP-Boc)

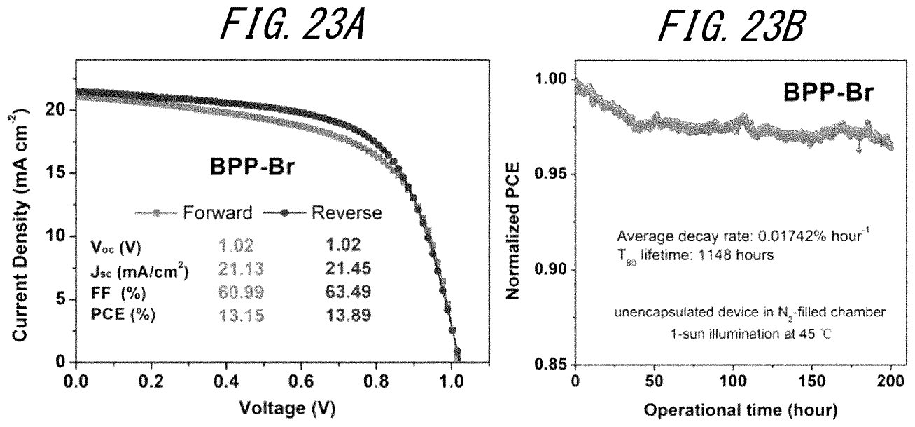

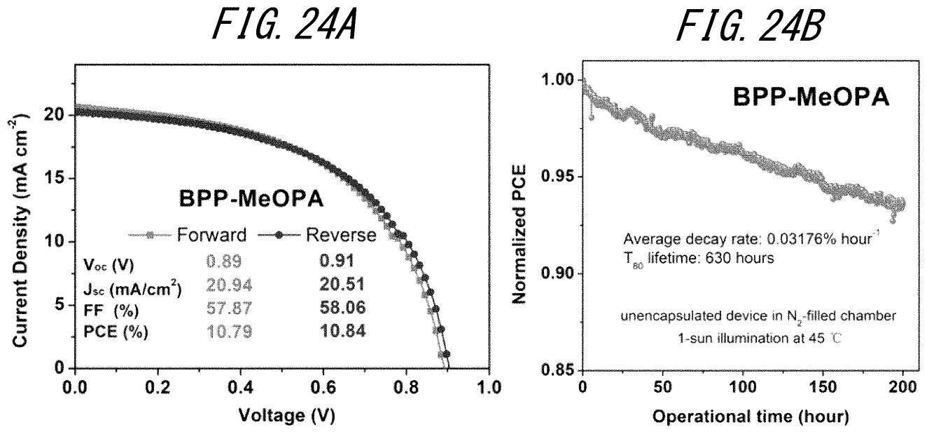

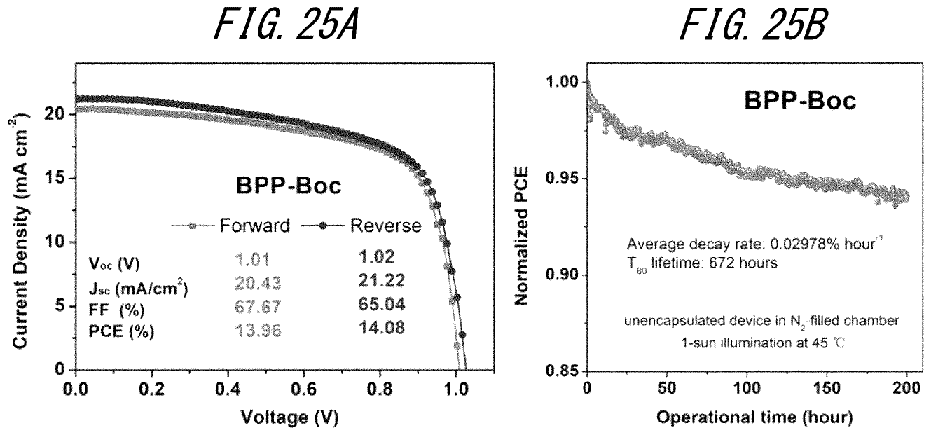

The inverted planar PSCs using BPP—Br, BPP-MeOPA or BPP-Boc instead of SA-BPP were prepared as in the same manner as described above to evaluate photovoltaic performance, and operational stability. FIGS. 23A, 24A, and 25A plot the J-V curves of the small-sized PSCs with an aperture area of 0.1 cm² based on the different hole-selective contacts, and FIGS. 23B, 24B, and 25B show the results of operational stability tests of the 0.1 cm² inverted PSCs in a N₂-filled chamber at 45° C. based on the different hole-selective contacts.

1. A hole-selective contact material comprising: a polycyclic aromatic hydrocarbon compound,
wherein the polycyclic aromatic hydrocarbon compound comprises a structure having a plurality of aromatic hydrocarbon rings, each of the aromatic hydrocarbon rings being fused with at least one other of the aromatic hydrocarbon rings, and at least one anchor group attached to the structure directly or via a linking group.

2. The hole-selective contact material according to claim 1, wherein the anchor group is selected from the group consisting of a carboxylic acid group, a carboxylate group, a cyanoacrylic acid group, a cyanoacrylate group, a pyridine group, a phosphonic acid group, a tetracyanate group, a perylene dicarboxylic acid anhydride group, a 2-hydroxybenzonitrile group, an 8-hydroxyquinoline group, a pyridine-N-oxide group, a 3-hydroxy-N-methylpyridinium group, a catechol group, a hydroxamate group, a sulfonic acid group, an acetylacetonate group, a boronic acid group, a nitro group, a tetrazol group, a rhodamine group, a rhodamine-3-acetic acid group, a salicylic acid group, an aldehyde group, a carbamate group, an unsubstituted or substituted amino group, an unsubstituted or substituted ammonium group, a halogen atom, and an imine group.

3. The hole-selective contact material according to claim 1, wherein the linking group is selected from the group consisting of an alkylene group, an alkenylene group, an alkinylene group, a cycloalkylene group, an arylene group, and a heteroarylene group.

4. The hole-selective contact material according to claim 1, wherein the structure having a plurality of aromatic hydrocarbon rings has an absorption edge from 350 to 550 nm.

5. The hole-selective contact material according to claim 4, wherein the structure having a plurality of aromatic hydrocarbon rings has an absorption edge from 400 to 500 nm.

6. The hole-selective contact material according to claim 1, wherein the plurality of aromatic hydrocarbon rings is 3 to 16 aromatic hydrocarbon rings.

7. The hole-selective contact material according to claim 1, wherein the plurality of aromatic hydrocarbon rings is a plurality of benzene rings.

8. The hole-selective contact material according to claim 1, wherein the structure is benzo[rst]pentaphene.

9. The hole-selective contact material according to claim 1, wherein the polycyclic aromatic hydrocarbon compound comprises at least one solubility-enhancing group.

10. The hole-selective contact material according to claim 9, wherein the solubility-enhancing group is selected from the group consisting of an alkyl group, an alkoxy group, an aryl group and an ester group.

11. A photoelectric conversion device comprising the hole-selective contact material according to claim 1.

12. The photoelectric conversion device according to claim 11, wherein the photoelectric conversion device is a perovskite solar cell.

13. A polycyclic aromatic hydrocarbon compound represented by formula (1): 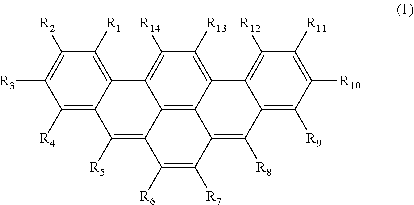
wherein
R₁, R₂, R₃, R₄, R₉, R₁₀, R₁₁, and R₁₂ are independently selected from hydrogen atoms, C1-C₁₂ alkyl and C1-C₁₂ alkoxy,
R₅ and R₅ are independently selected from Rₐ and -arylene-Rₐ, wherein Rₐ is carboxylic acid group, carboxylate group or carbamate group and
R₆, R₇, R₁₃, and R₁₄ are hydrogen atoms.
